SEQUENCE LISTING (18 sequences)

SEQ ID NO: 1 - DNA, 41 bp, Homo Sapiens
agccacaccagctatcaaaattctctaacgggcccttgcca

SEQ ID NO: 2 - DNA, 41 bp, Homo Sapiens
gccatggacatccacttccatagcccggccttccagcaccc

SEQ ID NO: 3 - DNA, 41 bp, Homo Sapiens
gttatgtgtgaagtcatttataatagatagtgttgataata

SEQ ID NO: 4 - DNA, 41 bp, Homo Sapiens
gttaacctgggagtgaactagacattaaaggggcagcatac

SEQ ID NO: 5 - DNA, 41 bp, Homo Sapiens
aaatcaagaagtgcatccatctgattctgaataattggtac

SEQ ID NO: 6 - DNA, 41 bp, Homo Sapiens
tctcagtagtggggttttgtcacctttactgttattattag

SEQ ID NO: 7 - DNA, 41 bp, Homo Sapiens
tgcctgcagcccaacagatggcagtgtgcttaacagctctg

SEQ ID NO: 8 - DNA, 41 bp, Homo Sapiens
tataccccgttaaaggaaacgatatagaatacgaatggtct

SEQ ID NO: 9 - DNA, 41 bp, Homo Sapiens
tagtttcaagtaaacatgtcgcagtgaagtttgttgtagag

SEQ ID NO: 10 - DNA, 41 bp, Homo Sapiens
ccctgtccgtccagactccgagagtctccgacgggtactac

SEQ ID NO: 11 - DNA, 41 bp, Homo Sapiens
caggctgcgaactttgcacctttacaccactccacgtgacc

SEQ ID NO: 12 - DNA, 41 bp, Homo Sapiens
caccacacacaaggcctgaacgatcaaaggctgaagagatg

SEQ ID NO: 13 - DNA, 41 bp, Homo Sapiens
cgctccaaacccgctgtgtcataccataccggatgtttacc

SEQ ID NO: 14 - DNA, 41 bp, Homo Sapiens
aatggtgagataggtatagaatagactgtctcagttcaaaa

SEQ ID NO: 15 - DNA, 40 bp, Homo Sapiens
ggcaaagaggggaggaagatgccaatagcttagattccca

SEQ ID NO: 16 - DNA, 41 bp, Homo Sapiens
gtttgggcatctgggggaaagccaggtttatgtaaatcaaa

SEQ ID NO: 17 - DNA, 41 bp, Homo Sapiens
agtctgaatttccagaaaacaatgatcattcaatggatcac

SEQ ID NO: 18 - DNA, 41 bp, Homo Sapiens
atctccaagtcaaagtttacctcaagttgggccaatccctg

(19) United States
(12) Patent Application Publication
(10) Pub. No.: US 2015/0119264 A1
(43) Pub. Date: Apr. 30, 2015

(54) DIAGNOSIS AND TREATMENT OF ALZHEIMER'S DISEASE

(71) Applicant: University College Cardiff Consultants Limited, Cardiff (GB)

(72) Inventors: Julie Williams, Cardiff (GB); Michael John Owen, Cardiff (GB)

(73) Assignee: University College Cardiff Consultants Limited, Cardiff (GB)

(21) Appl. No.: 14/252,153

(22) Filed: Apr. 14, 2014

Related U.S. Application Data
(63) Continuation of application No. 13/376,485, filed on Dec. 6, 2011, now abandoned.

(30) Foreign Application Priority Data
Jul. 3, 2009 (GB) 0911539.5
Jul. 31, 2009 (GB) 0913357.0
Oct. 3, 2009 (GB) 0917326.1

(51) Int. Cl. C12Q 1/68

(52) U.S. Cl. CPC C12Q 1/6883; C12Q 2600/156
USPC 506/9; 435/6.11; 506/7; 506/16; 506/17; 506/14

(57) ABSTRACT

The invention relates to novel variants that associate with Alzheimer's disease AD and their use in kits as a means for diagnosing AD; and also their use in nucleic acid molecules or cells/cell lines for identifying novel therapeutic, label of identification means.

This utility patent application is a Continuation of U.S. patent application Ser. No. 13/376,485 filed on Dec. 6, 2011, which is a national stage of international patent application no. PCT/GB2010/001208 filed on Jun. 22, 2010, which in turn claims priority from British Patent Application Ser. Nos. 0911539.5 filed on Jul. 3, 2009, 0913357.0 filed on Jul. 31, 2009, and 0917326.1 filed on Oct. 3, 2009, the disclosures of which are incorporated herein by reference in their entirety.

### TECHNICAL FIELD

The invention relates to the identification of variants in the CLU/APOJ, PICALM, ABCA7, CR1 or BIN1 gene loci or the MS4A gene cluster which are novel risk indicators for the development of Alzheimer's disease and also any single nucleotide polymorphisms (SNPs) in linkage disequilibrium therewith; the invention also relates to a method for diagnosing the risk of developing, or the existence of, Alzheimer's disease which involves assaying for the aforementioned variants in the CLU/APOJ, PICALM, ABCA, CR1 or BIN1 gene loci or the MS4A gene cluster; and the invention also relates to novel screening tools comprising the aforementioned loci or cluster.

### BACKGROUND OF THE INVENTION

Alzheimer's disease (AD) is the most common form of dementia, it is highly heritable (heritability of up to 76%) but genetically complex. Neuropathologically, the disease is characterised by extracellular senile plaques containing β-amyloid (Aβ) and intracellular neurofibrillary tangles containing hyperphosphorylated τ protein. Four genes have been definitively implicated in its aetiology. Mutations of the amyloid precursor protein (APP) gene and the presenilin 1 and 2 genes (PSEN1, PSEN2) cause rare, Mendelian forms of the disease, usually with an early-onset. However, in the more common form of AD, only apolipoprotein E (APOE) has been established unequivocally as a susceptibility gene. Aiming to identify novel AD loci, a small number of genome-wide association studies (GWAS) have been conducted prior to the present study. All have identified strong evidence for association to APOE, but less convincing evidence implicating other genes¹⁻⁸. This outcome is consistent with the majority of findings from GWAS of other common phenotypes.

Indeed, the lack of success of the afore GWAS has created a prejudice in the art which teaches away from GWAS as a way of identifying any novel AD loci, other than APOE. Despite this thinking we established a collaborative consortium from Europe and the USA from which we were able to draw upon a combined sample of up to 19,000 subjects (before quality control) and conducted a two-stage study. In Stage 1, 14,639 subjects were genotyped on Illumina platforms. 5,715 samples were genotyped for the present study using the Illumina 610-quadchip⁹; genotypes for the remaining subjects were either made available to us from population control datasets or through collaboration and were genotyped on the Illumina HumanHap550 or the HumanHap300 BeadChips. Prior to association analysis, all samples and genotypes underwent stringent quality control, which resulted in the elimination of 53,383 autosomal SNPs and 2,850 subjects. Thus, in Stage 1, we tested 529,205 autosomal SNPs for association in up to 11,789 subjects (3,941 AD cases, 7,848 controls of which 2,078 were elderly screened controls, see Table S1).

In addition to the known association with the APOE locus, GWA analysis identified two novel loci at a genome-wide level of significance (see FIG. 1).

One of the novel SNPs, rs11136000, is located within an intron of clusterin (CLU, also known as APOJ) on chromosome 8 (p=1.4×10⁻⁹, OR=0.840); the other SNP, rs3851179, is 88.5 kb 5′ to phosphatidylinositol-binding clathrin assembly (PICALM) on chromosome 11 (p=1.9×10⁻⁸, OR=0.849). Notably, therefore, neither SNP maps to a coding site and so is responsible for changes in an identified protein. They may therefore produce their effect at the level of gene expression, temporally or spatially, which, in turn, may affect transcription levels.

The afore statistical data provides compelling evidence that the two novel loci identified herein and their corresponding SNPs associate, with extremely high levels of probability, with Alzheimer's disease. This means that these SNPs can be used as powerful predicative tools for diagnosing the likelihood of developing Alzheimer's disease or the existence of Alzheimer's disease in the early stages of the condition.

A stage 2 sample (2,023 AD cases, 2,340 controls) provided further support for the association of these novel loci (CLU combined p=8.5×10⁻¹⁰, OR=0.86; PICALM combined p=1.3×10⁻⁹, OR=0.86).

In a preliminary attempt to attribute the source of the association to a functional variant, we used publicly available data to identify additional SNPs at each locus that were correlated through linkage disequilibrium (LD) with either of the afore novel SNPs or that might plausibly have functional effects. These markers were genotyped through the stage 2 sample. A synonymous SNP (rs7982) in the CLU gene was in strong LD (r²=0.95) with the genome-wide significant (GWS) SNP and showed a similar level of evidence for association with AD in the whole sample (meta-p=8×10⁻¹⁰; stage 1 genotypes were imputed). This SNP is in exon 5 of the CLU gene, which codes for part of the beta chain of the protein and may influence a predicted exon splicing enhancer. Several potentially functional SNPs were identified at the PICALM locus. Of these, two showed good evidence for association; rs561655, which is within a putative transcription factor binding site and rs592297, which is a synonymous SNP in exon 5 of the gene that may influence a predicted exon splicing enhancer.

The predominant form of clusterin is a secreted heterodimeric glycoprotein of 75-80 kDa. The single copy gene spans about 16 kb on chromosome 8p21-p12 and encodes an mRNA of approximately 2 kb that translates into a 449 amino acid primary polypeptide chain. Clusterin is expressed in all mammalian tissues and there is strong evidence that clusterin levels are elevated in a number of pathological conditions involving injury or chronic inflammation of the brain. In Alzheimer's disease brain, clusterin expression is reported to be increased in affected cortical areas and is present in amyloid plaques and in the cerebrospinal fluid of AD cases.

Clusterin is a multi-functional molecule. It interacts with the soluble form of Aβ in animal models of disease and binds soluble Aβ in a specific and reversible manner, forming complexes that have been shown to cross the blood-brain barrier. Interestingly, ApoE also appears to act as a molecular chaperone for Aβ and influences when it aggregates and deposits as well as influencing Aβ conformation and toxicity. In a similar way, clusterin appears to regulate both the toxicity and conversion of Aβ into insoluble forms. Furthermore, ApoE and clusterin have been shown to cooperate in suppressing Aβ deposition and ApoE and clusterin may critically modify Aβ clearance at the blood brain barrier, suggesting a role for clusterin in the amyloidogenic pathway. Levels of ApoE protein appear to be proportional to APOE-ε4 allele dose levels, i.e. expression levels are reduced in ε4 homozygotes compared with heterozygotes. Conversely, clusterin levels are increased in proportion to APOE-ε4 allele dose suggesting an induction of clusterin in individuals with low ApoE levels. Thus, the strong statistical evidence for the involvement of this gene in AD has additional support in terms of biological functionality.

The second gene locus to show compelling evidence for association with AD is PICALM (phosphatidylinositol-binding clathrin assembly protein; also known as CALM: clathrin assembly lyphoid-myeloid leukaemia gene). PICALM is ubiquitously expressed in all tissue types with prominent expression in neurons, where it is non-selectively distributed at the pre- and post-synaptic structures. It has been shown that PICALM is involved in clathrin-mediated endocytosis (CME), an essential step in the intracellular trafficking of proteins and lipids such as nutrients, growth factors and neurotransmitters. Of relevance to AD, PICALM appears to be involved in directing the trafficking of VAMP2. VAMP2 is a SNARE protein that plays a prominent role in the fusion of synaptic vesicles to the presynaptic membrane in neurotransmitter release, a process that is crucial to neuronal function. AD brains show a reduced number of synapses and stereological and biochemical analysis has shown that this reduction in synaptic density correlates with cognitive defects better than the accumulation of plaques. More recent analysis indicates synapses within AD brains may be dysfunctional even before they visibly degenerate. Therefore, we can hypothesise that genetically directed changes in PICALM function result in perturbations at the synapse, possibly through synaptic vesicle cycling, thereby increasing risk for AD. Alternatively, PICALM could influence risk of AD through APP processing via endocytic pathways resulting in changes in Aβ levels. Cell culture experiments have shown that full length APP is retrieved from the cell surface by CME and inhibition of endocytosis reduces APP internalisation and reduces Aβ production and release. Increased synaptic activity is known to lead to the elevated endocytosis of synaptic vesicle proteins and Cirrito et al. have since provided evidence in vivo that the increased CME, triggered by increased synaptic activity, drives more APP into endocytotic compartments resulting in an increase of Aβ production and release. Thus, as for clusterin, the strong statistical evidence for the involvement of PICALM in AD has support in terms of biological functionality.

We also tested whether the observed number of significant associations observed in the GWAS exceeded what would be expected by chance. Having removed SNPs within the APOE, CLU and PICALM loci (see Methods) we focused on those which showed most evidence for association (p<1×10⁻⁵). Approximately 13 independent signals were observed; less than 4 would be expected by chance (p=7.5×10⁻⁶). Table 2 shows the loci implicated and provides strong evidence for association with the complement receptor 1 (CR1) gene. Also noteworthy is the bridging integrator 1 (BIN1) gene, which produces a protein involved in synaptic vesicle endocytosis. These data thus provide strong evidence that these genes are associated with AD.

We sought to test those SNPs which showed the most promising evidence for association with AD in an independent sample comprising 3,262 AD cases and 5,064 controls which included the stage 2 samples previously discussed⁹. SNPs that showed P<1×10⁻⁵ from a meta-analysis of four GWAS datasets (a combined sample of up to 6,978 cases, 13,903 controls) were selected for genotyping (see Methods). For SNPs that showed evidence for association in the independent sample (P<0.05) additional consortia were approached so to combine all available AD GWAS data in an inverse variance weighted meta-analysis (see Methods). Four SNPs showed GWS evidence for association with AD (see Table 3); rs3764650 within ABCA7 (P=7.3×10⁻¹⁰, OR=1.22), rs670139 at the MS4A gene cluster (P=1.2×10⁻⁹, OR=1.12), rs744373 at the BIN1 locus (P=2.1×10⁻¹², OR=1.14) and rs3818361 at the CR1 locus (P=1.9×10⁻¹², OR=1.17)¹⁰

Variant rs3764650 is located in intron 13 of the ATP-binding cassette, sub family A, member 7 (ABCA7) gene (FIG. 6). ABCA7 encodes an ATP-binding cassette (ABC) transporter: the ABC transporter superfamily has roles in transporting a wide range of substrates across cell membranes. ABCA7 is highly expressed in brain, particularly in hippocampal CA1 neurons and in microglia. ABCA7 is involved in the efflux of lipids from cells to lipoprotein particles: the main lipoprotein in brain is APOE followed by CLU, thus ABCA7 may have a role in modulating their effects, although no evidence for genetic interactions was observed here (see Table S13). In addition ABCA7 has been shown to regulate APP processing and inhibit β-amyloid secretion in cultured cells overexpressing AP.

The genes in the MS4A cluster on chromosome 11 (chr11: 59,570,863-59,863,681 (Build GRCh37/hg19)) (FIG. 6) have a common genomic structure with all other members of the family, including transmembrane domains, indicating that they are likely to be part of a family of cell surface proteins. MS4A2 encodes the beta subunit of high affinity IgE receptors. The remaining genes in the LD block, including PLAC1L have no known specific functions.

BIN1 (also known as AMPH2, amphiphysin isoform 2) along with PICALM, functions in receptor-mediated endocytosis (RME). BIN1 expression is not brain specific, but there are several isoforms with enriched expression in brain. Knockdown of mammalian BIN1 delays endosome recycling and deletion mutants of the single amphiphysin isoform (AMPH1/AMPH2) orthologue in C. elegans (Amph-1) show defective endosome recycling.

CR1 is predominantly involved in adaptive immunity and is abundantly expressed on red blood cells, especially on intravascular erythrocytes and has been detected on neurons. CR1 mediates cellular binding to particles and immune complexes where it recognises that complement has been activated. CR1 can act as a negative regulator of the complement cascade, mediate immune adherence and phagocytosis and inhibit both the classical and alternative complement pathways. Markers of inflammation have been associated with AD previously and inflammatory processes proposed as pathogenic contributors. These data showing the involvement of several putative inflammatory genes (CLU, CR1, ABCA7 and possibly genes in the MS4A cluster) in AD, support a primary role for inflammatory processes in disease development.

### SUMMARY OF THE INVENTION

According to a first aspect of the invention there is therefore provided a method for screening for, or diagnosing the likelihood of developing, or the existence of, Alzheimer's disease comprising:
(a) providing a tissue sample which has been extracted from a human body of an individual to be tested wherein the tissue sample contains at least a loci containing the clusterin (CLU, also known as APOJ) gene on chromosome 8;
(b) examining said locus in order to identify whether SNP rs11136000 is present; and
(c) where SNP rs11136000 is present concluding that the individual from whom the sample has been extracted is likely to develop, or is suffering from, Alzheimer's disease.

According to a second aspect of the invention there is provided a method for screening for, or diagnosing the likelihood of developing, or the existence of, Alzheimer's disease comprising:
(a) providing a tissue sample which has been extracted from a human body of an individual to be tested wherein the tissue sample contains at least a loci containing the PICALM gene on chromosome 11;
(b) examining said locus in order to identify whether SNP rs3851179 is present; and
(c) where SNP rs3851179 is present concluding that the individual from whom the sample has been extracted is likely to develop, or is suffering from, Alzheimer's disease.

Additionally, or alternatively either of the above methods may be practised by substituting reference to the the clusterin (CLU, also known as APOJ) gene on chromosome 8 or the PICALM gene on chromosome 11 for either the complement receptor 1 gene (CR1) on chromosome 1, the bridging integrator/gene (BIN1) on chromosome 2, the ATP-binding cassette, sub family A, member 7 (ABCA7) on chromosome 19 or the chromosome 11 membrane-spanning 4A (MS4A) gene cluster and examining said loci to see if any one of the following SNPs is present rs1408077, rs6701713 or rs3818361 (for CR1); rs7561528 or rs744373 (for BIN1); rs3764650 (for ABCA7), and rs670139, rs610932, rs676309, rs667897, rs662196, rs583791 or rs1562990 for the MS4A gene cluster; and where the said SNP is present concluding that the individual from whom the sample has been extracted is likely to develop, or is suffering from, Alzheimer's disease.

According to a third aspect of the invention there is provided a method for screening for, or diagnosing the likelihood of developing, or the existence of, Alzheimer's disease comprising:
(a) providing a tissue sample which has been extracted from a human body of an individual to be tested wherein the tissue sample contains at least a locus containing the clusterin (CLU), also known as APOJ, gene on chromosome 8 or the PICALM gene on chromosome 11;
(b) examining said locus in order to identify whether SNP rs11136000 or rs3851179 is present; and
(c) where SNP rs11136000 or rs3851179 is present concluding that the individual from which the sample has been extracted is likely to develop, or is suffering from, Alzheimer's disease.

In any of the afore aspects of the invention reference to SNP rs11136000 is reference to a base change of T to C in intron 3 of the clusterin gene (transcript variant 1; NM_{—}001831.2; transcript variant 2 NM_{—} 203339.1; transcript variant 3 NM_{—} 001171138.1) at position chr8:27520436-27520436 (Build NCBI36/hg18) (See Table S16). The mapping of this SNP is shown in FIG. 2.

In any of the afore aspects of the invention reference to SNP rs3851179 is reference to a T to C base change 85.5 kb upstream of the PICALM gene (transcript variant 1; NM_{—}007166.2; transcript variant 2; NM_{—} 001008660.1) at position chr11: chr11:85546288-85546288 (Build NCBI36/hg18) (See Table S16). The mapping of this SNP is shown in FIG. 3.

In any of the afore aspects of the invention reference to SNP rs1408077 is reference to a base change of C to A in intron 38 of the of the CR1 gene (transcript variant F; NM_{—}000573.3) at position chr1:205870764-205870764 (Build NCBI36/hg18) (See Table S16).

In any of the afore aspects of the invention reference to SNP rs6701713 is reference to a base change of G to A in intron 31 of the of the CR1 gene (transcript variant F; NM_{—}000573.3) at position chr1:205852912-205852912 of the (Build NCBI36/hg18) (See Table S16).

In any of the afore aspects of the invention reference to SNP rs3818361 is reference to a base change of G to A in intron 29 of the of the CR1 gene (transcript variant F; NM_{—}000573.3) at position chr1:205851591-205851591 (Build NCBI36/hg18) (See Table S16).

In any of the afore aspects of the invention reference to SNP rs7561528 is reference to a base change of G to A 24.8 kb upstream of the BIN1 gene (transcript variant 1; NM_{—}139343.1; transcript variant 6; NM_{—} 139348.1; transcript variant 10; NM_{—}139351.1; transcript variant 2; NM_{—} 139344.1; transcript variant 3; NM_{—}139345.1; transcript variant 8; NM_{—} 004305.2; transcript variant 9; NM_{—} 139350.1; transcript variant 7; NM_{—}139349.1; transcript variant 5; NM_{—} 139347.1; transcript variant 4; NM_{—}139346.1) at position chr2: 127606107-127606107 (Build NCBI36/hg18) (See Table S16).

In any of the afore aspects of the invention reference to SNP rs744373 is reference to a base change of A to G 29.8 kb upstream of the BIN1 gene (transcript variant 1; NM_{—}139343.1; transcript variant 6; NM_{—} 139348.1; transcript variant 10; NM_{—}139351.1; transcript variant 2; NM_{—} 139344.1; transcript variant 3; NM_{—}139345.1; transcript variant 8; NM_{—} 004305.2; transcript variant 9; NM_{—} 139350.1; transcript variant 7; NM_{—}139349.1; transcript variant 5; NM_{—} 139347.1; transcript variant 4; NM_{—}139346.1) at position chr2:127611085-127611085 (Build NCBI36/hg18) (See Table S16).

In any of the afore aspects of the invention reference to SNP rs3764650 is reference to a base change of T to G in intron 13 of the of the ABCA7 gene (NM_{—}019112.3) at position chr19:1046520-1046520 (Build GRCh37/hg19) (See Table S16). The mapping of this SNP is shown in FIG. 6.

In any of the afore aspects of the invention reference to SNP rs1562990 is reference to a base change of C to A 25 kb upstream of the MS4A4A gene (transcript variant 2; NM_{—}148975.1) at position chr11:60023087-60023087 (Build GRCh37/hg19) (See Table S16).

In any of the afore aspects of the invention reference to SNP rs667897 is reference to a base change of A to G 2 kb upstream of the MS4A6A gene (transcript variant 1; NM_{—}152852.1; transcript variant 3; NM_{—}152851.1) at position chr11:59936979-59936979 (Build GRCh37/hg19) (See Table S16).

In any of the afore aspects of the invention reference to SNP rs676309 is reference to a base change of A to G 4 kb upstream of the MS4A4E gene at position chr11:60001573-60001573 (Build GRCh37/hg19) (See Table S16).

In any of the afore aspects of the invention reference to SNP rs583791 is reference to a base change of G to A in intron 3 of the MS4A6A gene (transcripts 1; NM_{—}152852.1; transcript 2; NM_{—}022349.2; transcript 3; NM_{—}152851.1) at position chr11:59947252-59847252 (Build GRCh37/hg19) (See Table S16).

In any of the afore aspects of the invention reference to SNP rs662196 is reference to a base change of G to A in intron 5 of the MS4A6A gene (transcripts 1; NM_{—}152852.1; transcript 2; NM_{—}022349.2; transcript 3; NM_{—}152851.1) at position chr11:59942757-59942757 (Build GRCh37/hg19) (See Table S16).

In any of the afore aspects of the invention reference to SNP rs610932 is reference to a base change of A to C in the 3 prime untranslated region of the MS4A6A gene (transcripts 1; NM_{—}152852.1; transcript 2; NM_{—}022349.2) at position chr11:59939307-59939307 (Build GRCh37/hg19) (See Table S16). The mapping of this SNP is shown in FIG. 6.

In any of the afore aspects of the invention reference to SNP rs670139 is reference to a base change of C to A 21 kb upstream of the MS4A4E gene at position chr11: 59971795-59971795 (Build GRCh37/hg19) (See Table S16). The mapping of this SNP is shown in FIG. 6.

In any of the afore aspects of the invention the said methodology may be undertaken using any one or more of the following variants, including any combination thereof: SNP rs1136000; SNP rs3851179; SNP rs1408077; SNP rs6701713; SNP rs3818361; SNP rs7561528; SNP rs744373; SNP rs7982; SNP rs561655; SNP rs592297; SNP rs3764650; SNP rs1562990; SNP rs667897; SNP rs676309; SNP rs583791; SNP rs662196; SNP rs670139 and SNP rs610932.

Preferably, in any of the above aspects of the invention identifying either one, or more, of the said SNPs involves the use of conventional genetic screening techniques well known to those skilled in the art of genotyping. For example, and without limitation, the aforementioned methods may be practised by the use of suitably labelled oligonucleotides which upon binding to, and so detecting the SNP of interest, emit a detectable signal representative of the presence of said SNP. Methods for the design of such oligonucleotides are well known to those skilled in the art and routinely practised in the identification and labelling of DNA. Further, the information provided in Table S16 enables those skilled in the art to design such oligonucleotides in conventional manner.

In either of the aforementioned aspects of the invention said tissue sample may be amplified prior to performing step (b) above. More preferably still, said amplified tissue may be enzymatically fragmented prior to performing step (b) above.

In yet a further preferred method of the invention said complementary oligonucleotide may be attached or bound to a solid phase or substrate and said, optionally, amplified and fragmented tissue sample may be exposed to said solid phase prior to performing the detection step referred to in (b) above.

According to a further aspect of the invention there is provided a kit for performing any one or more of the aforementioned methods of the invention wherein the kit comprises at least one oligonucleotide that is complementary to least one of the aforementioned loci and which is either provided with a suitable label that emits a detectable signal upon binding to the SNP of interest or there is provided associated labelling means which can be used in combination with said oligonucleotide whereby binding of the oligonucleotide to the SNP of interest enables the labelling means to be used to detect the aforementioned binding and so produce a signal representative of the presence of said SNP.

In a preferred embodiment said oligonucleotide(s) is/are immobilised on a solid support such as, without limitation, a bead, array or substrate.

According to a further aspect of the invention there is provided a method for screening for, or diagnosing the likelihood of developing, or the existence of, Alzheimer's disease comprising:
(a) providing a tissue sample which has been extracted from a human body of an individual to be tested wherein the tissue sample contains at least a locus containing the clusterin (CLU), also known as APOJ, gene on chromosome 8 or the PICALM gene on chromosome 11;
(b) examining said locus in order to identify whether SNP rs11136000 and/or SNP rs7982 which is in linkage disequilibrium therewith is present, or rs3851179 and/or SNP rs561655 or rs592297 which is in linkage disequilibrium therewith is present; and
(c) where any one or more of the said SNPs is present concluding that the individual from which the sample has been extracted is likely to develop, or is suffering from, Alzheimer's disease.

In any of the afore aspect of the invention reference to SNP rs7982 is reference to a base change of A to G in exon 5 of the of the clusterin gene (transcript variant 1; NM_{—}001831.2) at position chr2chr8:27518398-27518398 of the March 2006 human reference sequence (NCBI Build 36.1) (See Table S16).

In any of the afore aspect of the invention reference to SNP rs561655 is reference to a base change of A to G 20.2 kb upstream of the PICALM gene (transcript variant 1; NM_{—}007166.2) at position chr11:85477927-85477927 of the March 2006 human reference sequence (NCBI Build 36.1) (See Table S16).

In any of the above aspect of the invention reference to SNP rs592297 is reference to a base change of C to T in exon 5 of the PICALM gene (transcript variant 1; NM_{—}007166.2) at position chr11:85403585-85403585 of the March 2006 human reference sequence (NCBI Build 36.1) (See Table S16).

According to a further aspect of the invention there is provided a nucleic acid molecule comprising any one or more of the following loci or cluster: CLU/APOJ, PICALM, ABCA7, CR1 or BIN1, MS4A including one or more of the following variants, including any combination thereof; SNP rs1136000; SNP rs3851179; SNP rs1408077; SNP rs6701713; SNP rs3818361; SNP rs7561528; SNP rs744373; SNP rs7982; SNP rs561655; SNP rs592297; SNP rs3764650; SNP rs1562990; SNP rs667897; SNP rs676309; SNP rs583791; SNP rs662196; SNP rs670139 and SNP rs610932.

According to a further aspect of the invention there is provided a research tool for identifying therapeutics, labelling or identification means comprising: at least one genetic locus wherein said locus comprises on or more of the loci referred to above, that is, CLU, PICALM, CR1, BIN1, ABCA7, or MS4A loci which associate with AD, and more preferably the SNPs mentioned above in connection therewith.

According to a further aspect of the invention there is provided a cell or cell-line comprising one or more of the genetic loci of the invention and one or more of the associated SNPs for use to test whether a potential therapeutic, label or identification means can be used to treat AD or label or identify a marker that associates with AD.

Most suitably the cell or cell line comprises a functional endocytic, apoptotic, complement or innate immune response pathway whose modulation, by a test substance, can be used to determine the efficacy of said substance.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The invention will now be exemplified with reference to the following Accession numbers and Figures wherein:

### Accession Numbers

GenBank: CLU isoform 1 mRNA, NM_{—}001831.2; CLU isoform 2 mRNA, NM_{—}203339.1; CLU isoform 3 mRNA, NM_{—}001171138.1; PICALM isoform 1 mRNA, NM_{—}007166.2; PICALM isoform 2 mRNA, NM_{—}001008660.1. CR1 isoform S mRNA, NM_{—} 000651.4; CR1 isoform F mRNA, NM_{—}000573.3; BIN1 isoform 6 mRNA, NM_{—} 139348.1; BIN1 isoform 10 mRNA, NM_{—}139351.1; BIN1 isoform 2 mRNA, NM_{—} 139344.1; BIN1 isoform 3 mRNA, NM_{—}139345.1; BIN1 isoform 8 mRNA, NM_{—} 004305.2; BIN1 isoform 1 mRNA, NM_{—}139343.1; BIN1 isoform 9 mRNA, NM_{—} 139350.1; BIN1 isoform 7 mRNA, NM_{—}139349.1; BIN1 isoform 5 mRNA, NM_{—} 139347.1; BIN1 isoform 4 mRNA, NM_{—}139346.1; ABCA7 mRNA, NM_{—}019112.3; PLAC1L mRNA, NM_{—}173801.3; MS4A6E mRNA NM_{—}139249.2; MS4A3 isoform 2 mRNA, NM_{—}001031809.1; MS4A3 isoform 1 mRNA, NM_{—} 006138.4; MS4A3 isoform 3 mRNA, NM_{—}001031666.1; MS4A2 isoform 2 mRNA, NM_{—}001142303.1; MS4A2 isoform 1 mRNA, NM_{—}000139.3; MS4A6A isoform 1 mRNA, NM_{—}152852.1; MS4A6A isoform 3 mRNA, NM_{—}152851.1; MS4A6A isoform 2 mRNA, NM_{—}022349.2; MS4A4A isoform 2 mRNA, NM_{—}148975.1; MS4A4A isoform 1 mRNA, NM_{—}024021.2.

### BRIEF DESCRIPTION OF THE DRAWINGS

FIG. 1 is a scatterplot of chromosomal position (x-axis) against −log₁₀ GWAS P-value (y-axis). The y-axis scale has been limited to 9.25 (p=5.6×10⁻¹⁰), although highly significant association was observed with SNPs in the vicinity of the APOE locus (e.g. rs2075650 with p=1.8×10⁻¹⁵⁷). The threshold for genome-wide significance (p≦9.4×10⁻⁸) is indicated by the dashed horizontal line. 761 SNPs with p≦1×10⁻³ lie above the unbroken horizontal line. The plot was produced using Haploview v4.0;

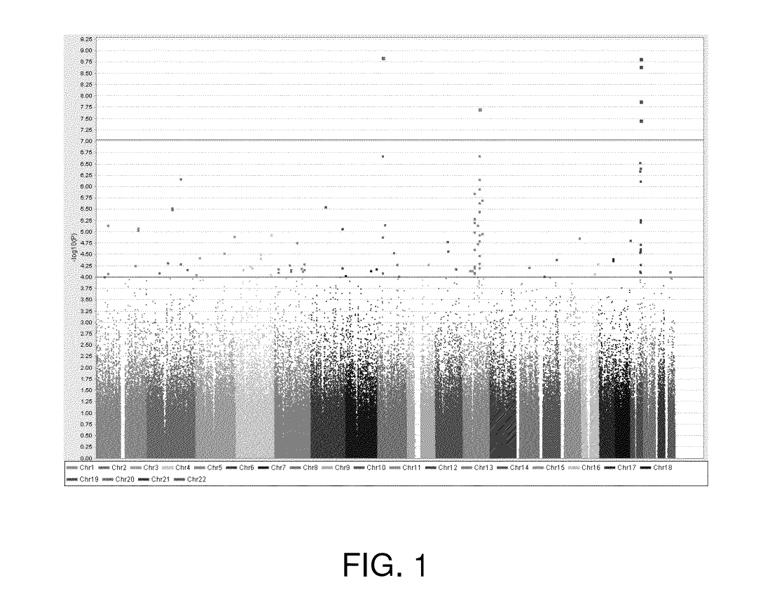

FIG. 2 is a schematic of the CLU gene based on CLU transcript variant 1. Chromosome position in megabases is shown at the top of the diagram. Gene schematic; horizontal arrow indicates direction of transcription, black boxes indicate gene exons, grey boxes indicate UTR, light grey boxes indicate transcript variant 2 specific UTR. The −log₁₀(P) of the SNPs genotyped in the GWA study is shown in chart graph with GWS SNP rs11136000 indicated. Meta p-values are indicated by dotted lines for GWS SNPs rs11136000 and rs7982. The D′ LD block structure of the CLU gene and surrounding region (chr8: 27,502,817-27,535,944), according to the CEU HapMap data, is provided at the bottom of the figure with lines indicating where each genotyped SNP is represented. Note, CLU transcript variant 2 does not contain exon 1 or the 5′UTR of transcript variant 1, also, in transcript variant 2, half of exon 2 is UTR;

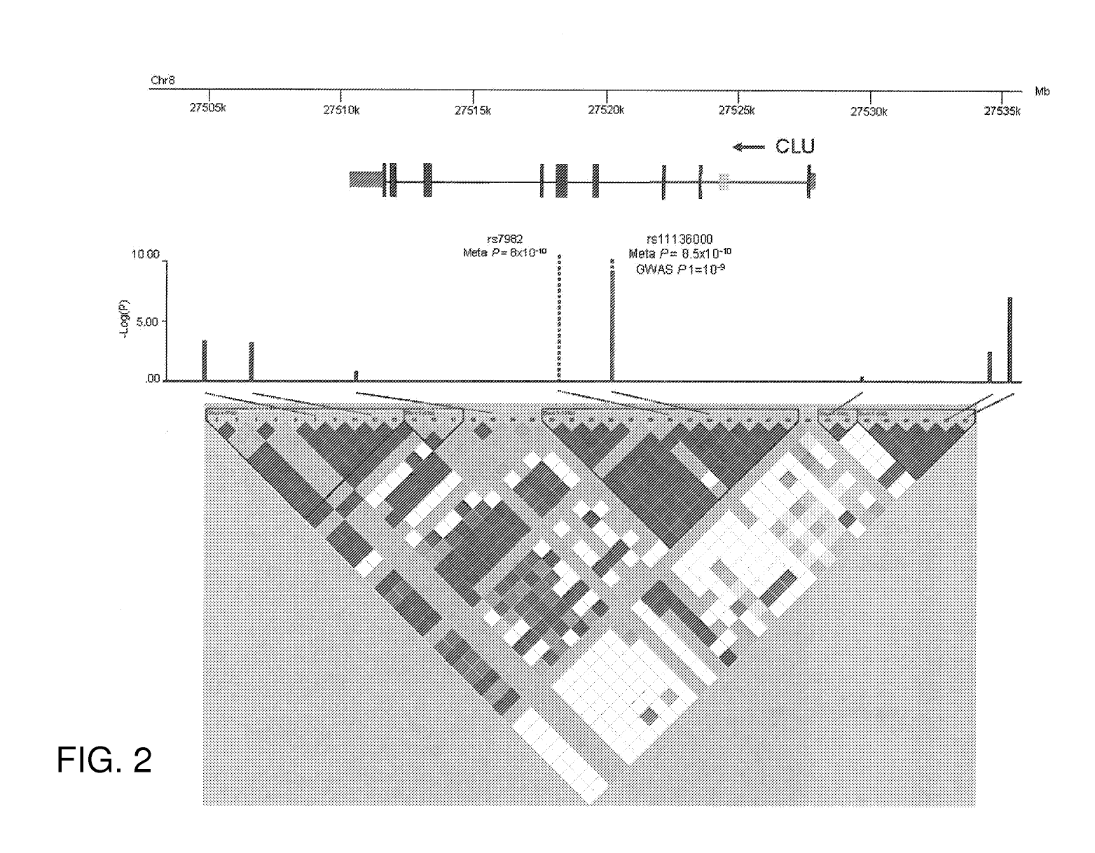

FIG. 3 is a schematic of the PICALM gene based on PICALM transcript variant 1. Chromosome position in megabases is shown at the top of the diagram. Gene schematic; horizontal arrow indicates direction of transcription, black boxes indicate gene exons, grey boxes indicate UTR, light grey boxes indicates a specific exon for transcript variant 2. The −log₁₀(P) of the SNPs genotyped in the GWA study is shown in chart graph with the GWS SNP rs3851179 highlighted in red. Meta p-values are indicated by dotted line for GWS SNP (rs3851179) plus putative functional variants (rs592297 and rs561655) that approach genome-wide significance. The D′ LD block structure of the PICALM gene and surrounding region (chr11: 85,326,133-85,605,600), according to the CEU HapMap data, is provided at the bottom of the figure;

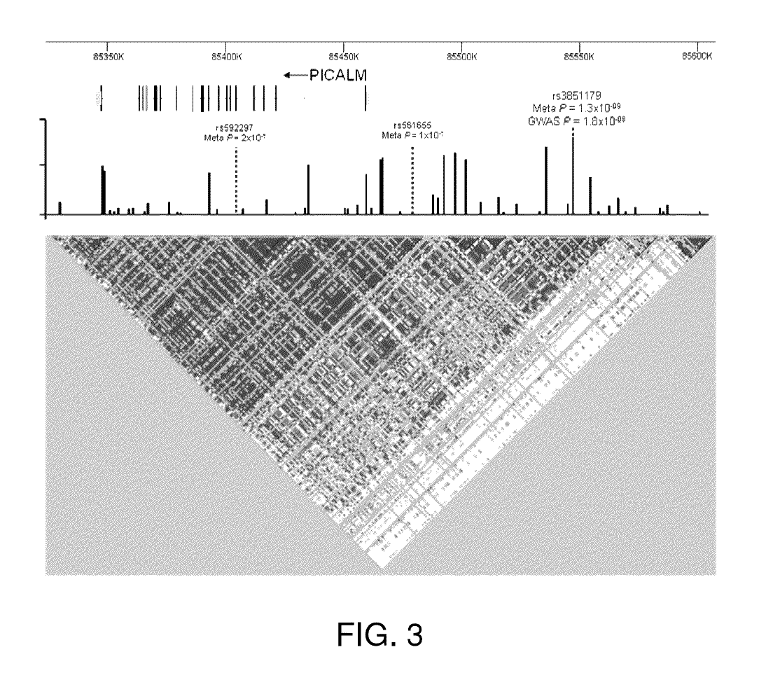

FIG. 4 shows an example of quantile-quantile plot when comparing different sets of controls. In this example, screened controls from the UK and Ireland, genotyped on the Illumina 610-quadchip are compared with controls from the 1958 British birth cohort, genotyped on the Illumina HumanHap550. The observed association χ² test statistics (y-axis) have been plotted against those expected under the null expectation (x-axis). The y-axis was limited at 30 although higher values were observed. The unbroken line of equality is shown. An exclusion χ2 threshold of 11 was employed (indicated by the dotted horizontal line);

FIG. 5A shows a quantile-quantile (Q-Q) plot of 529,205 observed genome-wide association χ² test statistics (y-axis) against those expected under the null expectation (x-axis) with the unbroken line of equality shown (λ=1.037); FIG. 5B shows the Q-Q plot after removal of 170 SNPs at the APOE locus, with 529,035 SNPs remaining (λ=1.037); and

FIG. 6 shows a schematic of the associated variants reported in reference to (a) the ABCA7 gene and (b) chromosomal region chr11:59.81 Mb-60.1 Mb harbouring members of the MS4A gene cluster. Chromosome positions are shown at the top of the schematics (Build GRCh37/hg19). Gene schematic: horizontal arrows indicate directions of transcription, black boxes indicate gene exons/UTR. The −Log₁₀(P) of the SNPs analyzed in Stage 1 are shown in chart graph. The Stage 3 inverse variance weighted meta-analysis P-values for SNPs rs3764650 (ABCA7), rs610932 (MS4A6A) and rs670139 (MS4A4E) are indicated. The non-synonymous SNP rs3752246 at the ABCA7 locus genotyped in the GERAD2 sample is also indicated (D′=0.89, r²=0.36 with rs3764650). The D′ LD block structure of the ABCA7 gene plus surrounding region, and chr11:59.81 Mb-60.1 Mb according to the CEPH HapMap data, are provided at the bottom of each schematic with lines indicating where each SNP genotyped on the Illumina 610-quad chip is represented.

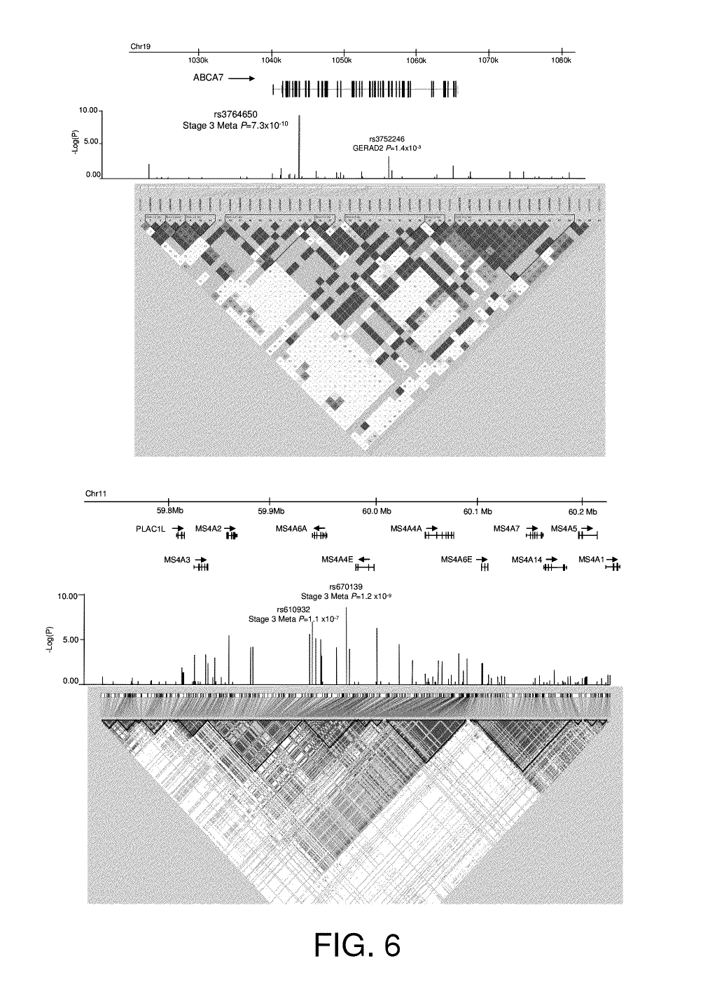

### DETAILED DESCRIPTION OF THE INVENTION

### Materials and Methods

### Sample Ascertainment and Diagnostic Criteria

The study comprised a Stage 1 ‘discovery sample’ of 4,957 AD cases and 9,682 controls and a Stage 2 ‘follow-up sample’ of 2,023 AD cases and 2,340 controls. Individuals included in this study were drawn from Europe and the United States. All individuals were of Caucasian ancestry. All AD cases met criteria for either probable (NINCDS-ADRDA, DSM-IV) or definite (CERAD)

AD. This study used ‘elderly screened controls’ and ‘population controls’. Elderly controls were screened for cognitive decline or neuropathological signs of AD. ‘Population controls’ were drawn from large existing cohorts with available genome-wide association data.

Stage 1 Discovery Sample: The discovery sample included 4,113 cases and 1,602 elderly screened controls genotyped at the Sanger Institute on the Illumina 610-quad chip, referred to collectively hereafter as the 610 group. These samples were recruited by the Medical Research Council (MRC) Genetic Resource for AD (Cardiff University; Institute of Psychiatry, London; Cambridge University; Trinity College Dublin), the Alzheimer's Research Trust (ART) Collaboration (University of Nottingham; University of Manchester; University of Southampton; University of Bristol; Queen's University Belfast; the Oxford Project to Investigate Memory and Ageing (OPTIMA), Oxford University); Washington University, St Louis, United States; MRC PRION Unit, University College London; London and the South East Region AD project (LASER-AD), University College London; University of Bonn, Germany and the National Institute of Mental Health (NIMH) AD Genetics Initiative. These data were combined with data from 844 AD cases and 1,255 elderly screened controls ascertained by the Mayo Clinic, Jacksonville, Fla.; Mayo Clinic, Rochester, Minn.; and the Mayo Brain Bank, which were genotyped using the Illumina HumanHap300 BeadChip. These samples were used in a previous GWAS of AD⁴. A total of 6,825 population controls were included in stage 1. These were drawn from large existing cohorts with available GWAS data, including the 1958 British Birth Cohort (1958BC) (http://www.b58cgene.sgul.ac.uk), NINDS funded neurogenetics collection at Coriell Cell Repositories (Coriell) (see http://ccr.coriell.org/), the KORA Study¹¹, Heinz Nixdorf Recall Study^{12,13} and ALS Controls. The ALS Controls were genotyped using the Illumina HumanHap300 BeadChip. All other population controls were genotyped using the Illumina HumanHap550 Beadchip. Clinical characteristics of the discovery sample can be found in Table S1.

Stage 2 Follow-up Sample: The follow-up sample comprised 2,023 AD cases and 2,340 controls. Samples were drawn from the MRC genetic resource for AD, the ART Collaboration, University of Bonn, Aristotle University of Thessaloniki, a Belgian sample derived from a prospective clinical study at the Memory Clinic and Department of Neurology, ZNA Middelheim, Antwerpen¹⁴ and the University of Munich. Clinical characteristics of the follow-up sample can be found in Table S2.

Additional Stage 2 Samples; The additional stage2 sample comprised 1,239 AD cases and 2,724 controls which were used for genotyping of the ABCA7 and MS4A loci. Samples were drawn from the MRC genetic resource for AD, the ART Collaboration, University of Bonn, and Aristotle University of Thessaloniki. Clinical characteristics of the full stage 2 follow-up sample can be found in Table S3.

### DNA Extraction and Laboratory Quality Control

DNA was obtained from blood samples from each participant, by phenol/chloroform extraction, followed by precipitation in ethanol and storage in TE buffer (some DNA was extracted using Qiagen kits). Initial DNA concentrations were determined by UV spectrophotometry (μQuant microplate spectrophotometer, Bio-Tek®, Beds, UK) or NanoDrop™ (Thermo Scientific, DE, USA). The concentration of each sample was then determined using the PicoGreen® dsDNA Quantitation Reagent (Molecular Probes®, Eugene, Ore.) in a Labsystems Ascent Fluoroskan® (LifeSciences Int., Basingstoke, UK). Each sample was then diluted to 50 ng/ul and allowed to equilibrate at 4° C. for 48 h. DNA quality was assessed by agarose gel electrophoresis under standard conditions. Samples showing no evidence of degradation were then genotyped in a panel of 30 SNPs using the MassARRAY® and iPlex® systems (Sequenom®, San Diego, Calif.) following manufacturer's protocols. This allowed gender to be checked and permitted sample identity checks after re-arraying samples for GWAS.

Stage 1 Genotyping (610 group)

Genotyping was performed at the Sanger Institute, UK. All normalised samples passing quality control (QC) were re-arrayed for GWAS using a Biomek® FX Laboratory Automation Workstation (Beckman Coulter®, Inc., Fullerton, Calif.) into 96 well plate formats. 200 ng of input DNA per sample were used and prepared for genotyping using the Illumina Infinium™ system (Illumina® Inc., San Diego, Calif., USA). Manufacturer's protocols were followed throughout. Briefly, DNA was isothermally amplified overnight then enzymatically fragmented, alcohol precipitated and resuspended. In this study we used Illumina Human 610-Quad BeadChips (Illumina® Inc., San Diego, Calif., USA) which were prepared for hybridization in a capillary flow-through chamber. The amplified and fragmented DNA samples were hybridised to the bead chips using a Tecan robot and an enzymatic base extension used to confer allele specificity. The chips were subsequently stained and scanned using an iSCAN reader (Illumina® Inc., San Diego, Calif., USA) to detect fluorescence at each bead. Data were loaded into Beadstudio and final call reports containing X, Y, X-Raw and Y-Raw outputted. The Illuminus algorithm for cluster analysis was used for genotype calling¹⁵.

### Stage 1: Individual Quality Control

4,113 AD cases and 1,602 controls were genotyped on the Illumina 610-quad chip as part of this study (the 610 group). In addition, 844 AD cases and 8,080 controls previously genotyped using either the Illumina HumanHap550 or Illumina HumanHap300 were included in the analysis. These genotypes were generated as part of 7 different studies, making 8 separate groups in total: 1) 610; 2) Mayo; 3) 1958 birth cohort (Sanger); 4) 1958 birth cohort (T1DGC); 5) ALS control; 6) Coriell control; 7) Heinz Nixdorf Recall (HNR) study; 8) Kora. As we used genotype data from multiple sources, it was important to apply stringent QC filters, as differential genotyping error rates between groups could result in spurious associations when the data are combined^{16,17}. These filters were applied separately to each of these 8 groups to remove poorly performing samples using tools implemented in PLINK v1.05 (http://pngu.mgh.harvard.edu-purcell/plink)¹⁸. The specific QC thresholds applied and the breakdown of samples excluded by group are given in Tables S4 and S5, respectively.

We removed 1,469 individuals with missing genotype rates >0.01. We also applied a filter based on mean autosomal heterozygosity, excluding 578 individuals with values above or below empirically determined thresholds. 71 individuals with inconsistencies between reported gender and genotype-determined gender and 22 individuals with ambiguous genotype-determined gender were removed. All individuals passing these QC filters were examined for potential genetic relatedness by calculating identity by descent (IBD) estimates for all possible pairs of individuals in PLINK, and removing one of each pair with an IBD estimate ≧0.125 (the level expected for first cousins). IBD estimates were calculated using SNPs that were common to the lumina 610, 550 and 300 chips with a genotype missing data rate ≦0.01, Hardy-Weinberg P≧1×10⁻⁵ and a minor allele frequency ≧0.01. As a result, 506 individuals were excluded (note that this includes 311 individuals that were included in both the Coriell and ALS control group).

We also sought to detect non-European ancestry. To this end, genotype data from SNPs typed in all cohorts was merged with genotypes at the same SNPs from 210 unrelated European (CEU), Asian (CHB and JPT) and Yoruban (YRI) samples from the HapMap project. Subsequent to removing SNPs in extensive regions of linkage disequilibrium (chr5:44-51.5 Mb; chr6: 25-33.5 Mb; chr8: 8-12 Mb; chr11: 45-57 Mb)¹⁹, we further pruned SNPs if any pair within a 50-SNP window had r²>0.2. Genome-wide average identity by state (IBS) distance was calculated in PLINK between each pair of individuals in the resulting dataset, based on 57,966 SNPs (all with a genotype missing data rate ≦0.01, Hardy-Weinberg P≧1×10⁻⁵ and a minor allele frequency ≧0.01). The resulting matrix of IBS distances was used as input for classical multi-dimensional scaling (MDS) in R v2.7.1 (http://www.r-project.org). When the first two dimensions were extracted and plotted against each other, three clusters were observed corresponding to the European, Asian and Yoruban samples. Sixteen samples appeared to be ethnic outliers from the European cluster and were excluded from further analysis.

We assessed population structure within the data using principal components analysis (PCA) as implemented in EIGENSTRAT²⁰ to infer continuous axes of genetic variation. Eigenvectors were calculated based on the previously described LD-pruned subset of 57,966 SNPs common to all arrays. The EIGENSTRAT program also identifies genetic outliers, which are defined as individuals whose ancestry is at least 6 standard deviations from the mean on one of the top ten axes of variation. As a result, 188 outliers were identified and excluded. Following sample QC 3,941 AD cases and 7,848 controls were included in the analysis.

### Stage 1: SNP Quality Control

Only autosomal SNPs were included in this analysis. Individuals were genotyped on either the Rumina 610-quad as part of this project, or were previously genotyped on the Illumina HumanHap550 or the Illumina HumanHap300 array, and the genotypes made available to us. Note that SNPs had already been filtered out of some groups prior to inclusion in this study. Moreover, where different versions of the same array were used (e.g. HumanHap550v1 used to genotype the 1958 birth cohort (Sanger) cohort compared with the HumanHap550v3 array used to genotype the 1958 birth cohort (T1DGC)), only SNPs common to both versions were considered as present on that array. As such, SNPs included in our analysis fell into 4 different categories; 1) 266,714 SNPs common to all 3 arrays and genotyped in all individuals; 2) 202,516 SNPs common to the 610 and 550 arrays, but not present or without genotypes in individuals typed on the 300 array; 3) 7,744 SNPs common to the 610 and 300 arrays, but not present or without genotypes in individuals typed on the 550 array; 4) 105,614 SNPs with genotypes only in the 610 data (see Table S6).

We assessed the effects of different missing data rate and Hardy-Weinberg filters, aiming to remove poorly performing SNPs without excluding markers that may show genuine association with AD. For each of the 4 SNP categories, markers were excluded if they had a minor allele frequency (MAF)<0.01 or a Hardy-Weinberg P≦1×10⁻⁵, in either cases or controls. SNPs with a MAF ≧0.05 were excluded if they had a genotype missing rate of >0.03 in either cases or controls; for SNPs with a MAF between 0.01 and 0.05, a more stringent genotype missing rate threshold of 0.01 was employed. As a result of this basic SNP QC 43,542 SNPs were excluded.

Ten principal components (PCs) were extracted using EIGENSTRAT, as previously described. To determine if the PCs could assuage any population structure within our sample, we performed logistic regression tests of association with AD, sequentially including between 0 and 10 of the top PCs as covariates. The impact of including the PCs was evaluated by calculating the genomic control inflation factor, λ²¹. We found that including the first 4 PCs as covariates had the maximum impact on λ (see Table S7).

To minimise inter-chip and inter-cohort differences that could result in an inflation of type I error rate, minor allele frequencies were compared between controls in the different groups using logistic regression analysis, incorporating the top 4 PCs as covariates as previously described. Comparisons were only performed between individuals from the same geographical region (i.e. British Isles, Germany or USA) and included: 1) 1958 birth cohort (Sanger) versus 1958 birth cohort (T1DGC); 2) 1958 birth cohort (combined Sanger and T1DGC) versus 610 UK controls; 3) 1958 birth cohort (combined Sanger and T1DGC) versus ALS UK controls; 4) 610 UK controls versus ALS UK controls; 5) HNR cohort versus Kora cohort; 6) 610 German controls versus combined HNR and Kora cohort; 7) Mayo controls versus ALS US controls; 8) Coriell cohort versus combined Mayo and ALS US controls; 9) 610 US controls versus combined Mayo and ALS US controls; 10) 610 US controls versus Coriell cohort. Moreover, as a result of comparisons 2, 4, 6, 7, 9 and 10, elderly screened controls were compared with unscreened/population controls.

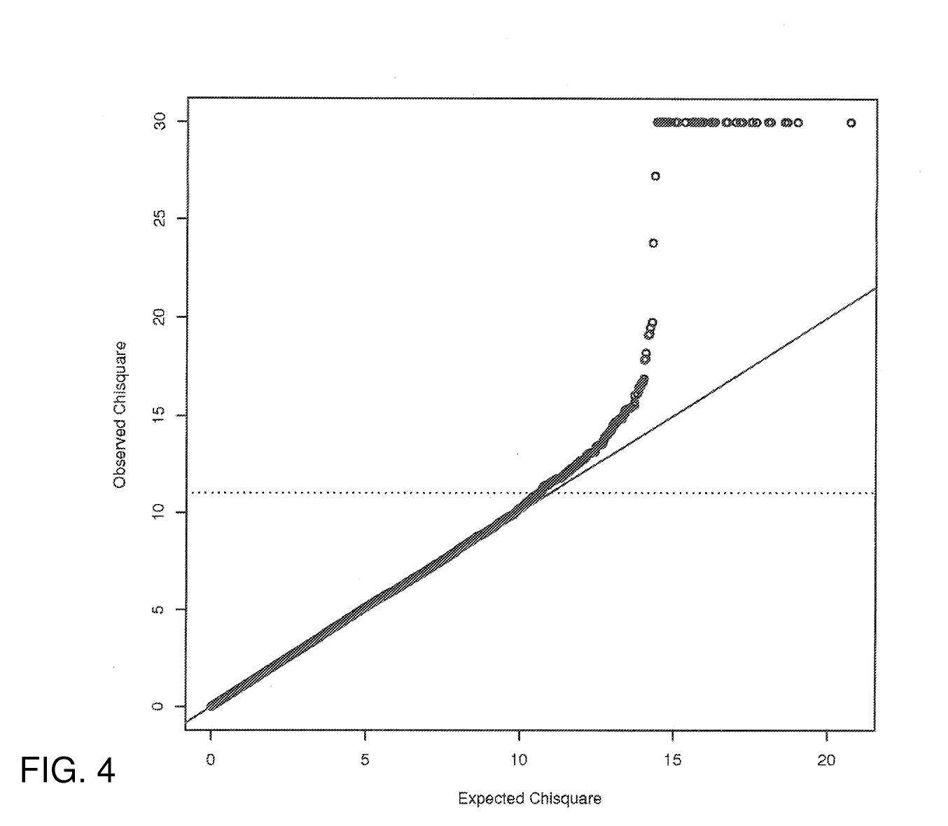

For each of the 4 categories of SNPs, a quantile-quantile (Q-Q) plot was produced for each cohort control comparison, and the significance threshold employed to exclude SNPs was based on where the observed χ² statistics departed from the null expectation (see Table S8 and FIG. 4). A further 9,828 SNPs were excluded as a result of these comparisons. Thus, a total of 529,218 SNPs were analysed for association with AD in this study.

### Stage 1 Statistical Analysis

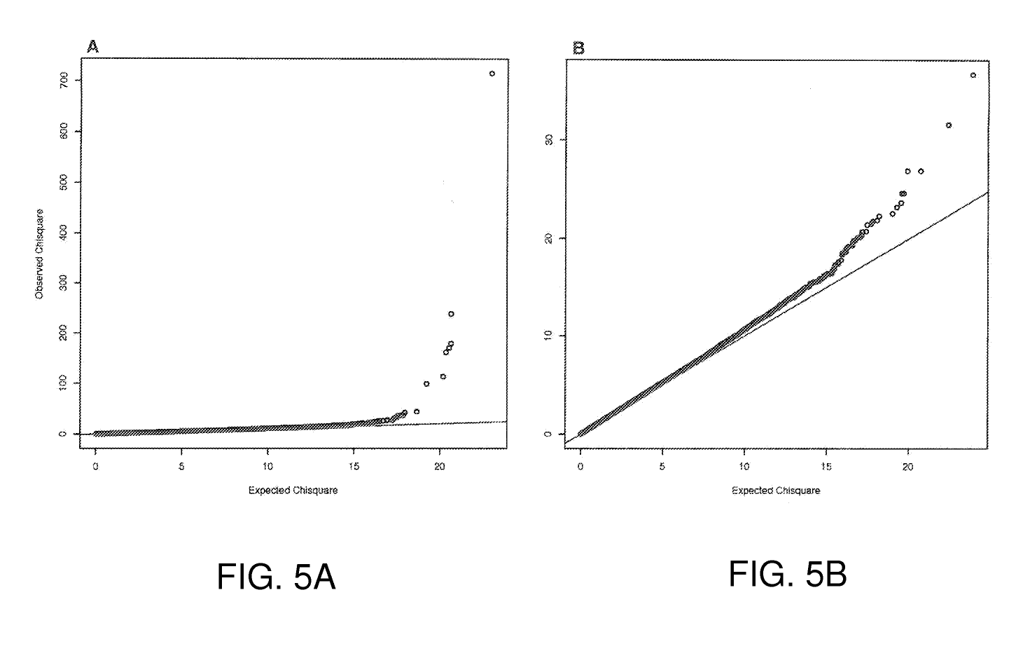

SNPs were tested for association with AD using logistic regression, assuming an additive model. Covariates were included in the logistic regression analysis to allow for geographical region and chip, i.e. to distinguish between 1) individuals from the British Isles, 2) individuals from Germany, 3) individuals from the US typed on the 610 or 550 chip, 4) individuals from the US typed on the 300 chip. It was not possible to include a covariate for each chip as only controls were genotyped on the 550 chip. Similarly, it was not possible to include a covariate for each of the 8 groups, as only two included both cases and controls (610 and Mayo groups). The first 4 PCs extracted from EIGENSTRAT were also included as covariates, as previously described. Following analysis, 130 cluster plots were visually inspected for SNPs with a p-value ≦1×10⁻⁴. Thirteen SNPs showing poorly formed clusters were excluded. Thus our analysis was based on 529,205 SNPs, and a conservative genome-wide significance threshold of 0.05/529205=9.4×10⁻⁸ was employed. Q-Q plots of the test results are shown in FIG. 5. The overall genomic control inflation factor, λ, was calculated to be 1.037. Results are shown for SNPs with a p-value ≦1×10⁻⁴ in Table S9. A breakdown of minor allele frequencies in cases and controls is shown for genome-wide significant SNPs in Table S10.

### Stage 2 Genotyping and Statistical Analysis

We genotyped SNPs in cases and controls from 5 European cohorts (described in Table S2 and S3). Genotyping was performed at Cardiff using the MassARRAY and iPlexGOLD systems (Sequenom, San Diego, Calif.) according to manufacturer's recommendations. All assays were validated prior to use, based on optimisation in 30 reference Centre d′Etude du Polymorphisme Humain (CEPH) parent-offspring trios. Sample plates contained cases, controls and blank samples. Quality control measures included independent double genotyping blind to sample identity and blind to the other rater, and where available comparison of our CEPH genotypes to those in the HapMap (www.hapmap.org). In addition, 231 individuals included in the GWAS were also genotyped on the Sequenom platform. We calculated the average concordance rate for the 7 SNPs typed on both platforms to be 99%. All genotyped SNPs had genotype call frequency rates >90% in the follow-up sample, and no SNPs had HWE P-value ≦0.05 in cases or controls. SNPs were tested for association with AD using logistic regression, assuming an additive model. Covariates were included in the logistic regression analysis to allow for each cohort, i.e. 1) Belgium, 2) MRC, 3) ART, 4) Bonn, 5) Greek.

### CLU and PICALM Meta-Analysis

We included genotype data from stages 1 and 2 in a meta-analysis for SNPs at the CLU and PICALM loci. In addition, we employed genotype data from the TGEN study, a publicly available AD GWAS dataset. This sample is comprised of 861 AD cases and 550 controls genotyped on the Affymetrix 500K chip. If a SNP of interest was not genotyped in our GWAS or the TGEN dataset, an attempt was made to impute genotypes in PLINK, using the 60 HapMap CEU founders as a reference panel. Only imputed SNPs with an information content metric value greater than 0.8 were included in analysis (see PLINK website). SNPs were tested for association with AD using logistic regression, assuming an additive model. Covariates were included in the logistic regression analysis to allow for geographical region and chip as in Stage 1 and for cohort as in Stage 2. Covariates included for the TGEN sample distinguished between samples from the Netherlands Brain Bank and samples from the USA. Results of the meta-analysis are shown in Table 1 and Table S11.

### ABCA7 and MS4A Meta-Analysis

### Discovery Meta-Analysis

We included genotype data from stages 1 and the full stage 2 sample in a meta-analysis for SNPs at the ABCA7 and MS4A loci. In addition, we employed genotype data from the TGEN (Translational Genomics Research Institute)²² and ADNI (Alzheimer's Disease Neuroimaging Initiative)²³ studies, both publicly available AD GWAS datasets, analysed in house for association to AD using a logistic regression assuming an additive model and including country of origin covariates for the TGEN study¹⁰. We first performed an inverse variance weighted fixed effects meta-analysis of the GERAD1, ADNI and TGEN datasets. The P-values from this meta-analysis were then combined with the publicly available P-values from the EADI1 (European Alzheimer's Disease Initiative Stage 1)²⁴ study using Fisher's combined probability test (note that odds ratios and variances for all SNPs genotyped in the EADI1 study were not publicly available, thus precluding an inverse variance weighted meta-analysis of all 4 GWAS). The combined analysis tested 496763 autosomal SNPs. These SNPs passed QC in our stage 1 sample and each of the ADNI and EADI1 GWA studies; 52391 of these SNPs were also genotyped and passed QC in the TGEN GWAS (which unlike the other studies employed the Affymetrix 500K array). In the combined analysis, 61 SNPs were associated with AD at P≦1×10⁻⁵ (see Table S14).

### Inverse Variance Weighted Meta-Analysis of all Available Data

For the five SNPs that showed evidence for association in our stage 2 sample (P<0.05), summary data (including odds ratios and variances) were obtained from the CHARGE (Cohorts for Heart and Aging Research in Genomic Epidemiology)²⁵ and EADI1 studies, such that all data (i.e., our stage 1 & 2, EADI1, ADNI, TGEN and CHARGE) were combined in an inverse-variance weighted fixed effects meta-analysis (total sample of up to 11607 cases and 31871 controls). Cochran's Q-test was performed and I² calculated to assess heterogeneity. Although the same direction of effect was observed in all studies analyzed for the CR1 SNP rs3818361, there was some evidence of heterogeneity (Cochran's Q test P=0.02, I²=65%). A random effects meta-analysis of this SNP was also performed for comparison (see Table S15).

### Secondary Analyses

We also tested the GWS SNPs for relationships with age at onset (AAO). To this end, age at onset (in years) was employed as the dependent variable in a linear regression analysis and an additive model was assumed. AAO data was available for 2,856 AD cases. Covariates were included in the logistic regression analysis to allow for geographical region and chip, i.e. to distinguish between 1) cases from the British Isles, 2) cases from Germany, 3) cases from the US typed on the 610 chip, 4) cases from the US typed on the 300 chip. Results are shown in Table S12.

In addition, we stratified our sample based on presence/absence of at least 1 APOE ε4 allele. We had APOE genotype data for 6045 individuals; our ε4-positive sample consisted of 2,203 AD cases and 632 controls; our ε4-negative sample consisted of 1,446 cases and 1,764 controls. We performed genome-wide tests for association with AD in each sub-sample, but no SNP achieved genome-wide significance (data not shown).

### Expected Number of Significant SNPs

We assessed our results to determine if we observed more significant SNPs at stage 1 than would be expected by chance. We first removed SNPs within 500 kb either side of risk SNPs, i.e. rs429358 (the APOE ε4 SNP), rs11136000 (CLU) and rs3851179 (PICALM). We thus excluded 170 “APOE” SNPs, 290 “CLU” SNPs and 257 “PICALM” SNPs. Of the 528,448 remaining SNPs we estimated 397,224.7 “independent” tests using the algorithm we described in (15). Of 16 SNPs significant at a significance level α=10⁻⁵ (excluding APOE, CLU and PICALM SNPs) we estimated 12.6 “independent” tests. We calculated mean (N*α=397224.7*10⁻⁵≈4.0) and variance (N*α*(1−α)=3.97) of the expected number of significant tests at α=10⁻⁵ level using the binomial distribution. Thus the probability of observing 12.6 significant tests is P=7.5×10⁻⁶.

### Linkage Disequilibrium Testing

Preliminary analysis of Novel GWS loci: To further explore relationships within the two associated gene regions (CLU and PICALM) we selected additional SNPs from each, which either showed some evidence for association at Stage 1 (p≦0.001) or were putative functional SNPs in linkage disequilibrium (D′>0.3) with the novel GWS hits (see Table S11). Predicted functional SNPs were identified using PupaSuite (http://bioinfo.cipf.es/pupasuite/www/index.jsp)²⁶. These were tested for association with AD in an independent sample and also checked for association in the TGEN GWAS. Four additional SNPs were selected from the CLU locus, one which showed some evidence at stage 1 (rs7012010; p=8×10⁻⁴) and three which had putative functional significance but were not typed in stage 1 (rs3087554, rs9331888 and rs7982, see Table S11). rs7012010 showed no evidence for association in the original Stage 2 sample, some evidence in the TGEN dataset (p=0.033) but did not reach GWS levels of significance in the meta-analysis (p=1×10⁻⁴). Two of the remaining SNPs showed no evidence for association, whereas the third rs7982, a synonymous SNP in strong LD (r²=1 in HapMap CEU individuals; r²=0.95 in extension sample) with the GWS SNP in clusterin, did show association in the original Stage 2 sample, with a similar magnitude of effect (meta p=8×10⁻¹⁰; stage 1 genotypes imputed).

We selected 8 additional SNPs for follow-up from the PICALM locus: half showed significant evidence for association in Stage 1 and half were chosen for their putative function (see Table S11). Three of the first four SNPs showed evidence for association in both the original Stage 2 and TGEN samples with the fourth showing association in the TGEN dataset alone. Producing combined p-values of: rs677909, p=3×10⁻⁸; rs541458, p=5×10⁻⁹; rs543293, p=1×10⁻⁸; and rs7941541 p=3×10⁻⁹, none of which exceeded that of the original GWS SNP. All of these SNPs were in high LD with the original GWS SNP. The only potentially functional SNPs which showed good evidence for association were rs561655 which is within a putative transcription factor binding site and rs592297 which is a synonomous SNP in exon 5 of the gene which may influence a putative exon splicing enhancer. However, neither of these SNPs showed the strength of evidence for association observed for rs3851179, the GWS SNP, producing meta p=1×10⁻⁷ and p=2×10⁻⁷, respectively.

### CONCLUSIONS

Our results provide compelling evidence that the genes described herein are true susceptibility genes for AD. However, a further striking implication of our findings is the support for additional disease mechanisms that go beyond Aβ accumulation. Three, and possibly four of the recently identified AD susceptibility loci (CLU, CR1, ABCA7 & the MS4A gene cluster) have known functions in the immune system, specifically the classical complement pathway. Furthermore, both PICALM and BIN1 are involved in clathrin-mediated endocytosis and APOE, CLU and ABCA7 in lipid processing.

TABLE 1
SNPs showing genome-wide significant association with AD in stage 1 of the GWAS. P-values in the original
stage 2 extension sample, the combined stage1&2 sample, and a meta-analysis of all available data (EADI1,
ADNI, TGEN and CHARGE) are also shown for the two SNPs unlinked to the APOE locus (highlighted in bold).

| | | | | | | | | Meta-analysis | | Meta-analysis Stage 1&2, | |
|---|---|---|---|---|---|---|---|---|---|---|---|
| | | | | | | Stage1: | Stage2: | Stage1&2: | | EADI1, ADNI, TGEN, | |
| | | | | | | 3941 cases | 2023 cases | 5964 cases | | CHARGE; 10368 cases, | |
| | | | Closest | Location | | 7848 controls | 2340 controls | 10188 controls | | 29147 controls | |
| | | | RefSeq | Relative | | P-value | P-value | P-value | OR | P-value | OR |
| SNP | Chr | MB | Gene | to Gene | MAF | (two-tailed) | (one-tailed) | (two-tailed) | (95% CI) | (two-tailed) | (95% CI) |
| rs2075650 * | 19 | 50.1 | TOMM40 | Intron | 0.15 | 1.8 × 10⁻¹⁵⁷ | | | | | |
| rs157580 | 19 | 50.1 | TOMM40 | Intron | 0.39 | 9.6 × 10⁻⁵⁴ | | | | | |
| rs6859 | 19 | 50.1 | PVRL2 | 3′UTR | 0.43 | 6.9 × 10⁻⁴¹ | | | | | |
| rs8106922 | 19 | 50.1 | TOMM40 | Intron | 0.40 | 5.4 × 10⁻³⁹ | | | | | |
| rs405509 | 19 | 50.1 | APOE | 5′ | 0.52 | 4.9 × 10⁻³⁷ | | | | | |
| rs10402271 | 19 | 50.0 | BCAM | 3′ | 0.32 | 1.5 × 10⁻²⁶ | | | | | |
| rs439401 † | 19 | 50.1 | APOE | 3′ | 0.36 | 2.7 × 10⁻²³ | | | | | |
| rs2927438 | 19 | 49.9 | BCL3 | 5′ | 0.21 | 3.0 × 10⁻¹¹ | | | | | |
| rs377702 | 19 | 50.1 | PVRL2 | Intron | 0.38 | 8.4 × 10⁻¹¹ | | | | | |
| rs11136000 | 8 | 27.5 | CLU | Intron | 0.40 | 1.4 × 10⁻⁹ | 0.017 | 8.5 × 10⁻¹⁰ | 0.86 | 8.7 × 10⁻¹⁹ | 0.86 |
| | | | | | | | | | (0.82-0.90) | | (0.84-0.89) |
| rs1048699 | 19 | 50.3 | NKPD1 | 3′ | 0.09 | 1.5 × 10⁻⁹ | | | | | |
| rs1114832 | 19 | 50.3 | LRRC68 | Intron | 0.09 | 2.2 × 10⁻⁹ | | | | | |
| rs1871047 | 19 | 50.0 | PVRL2 | Intron | 0.40 | 1.3 × 10⁻⁸ | | | | | |
| rs3851179 | 11 | 85.5 | PICALM | 5′ | 0.37 | 1.9 × 10⁻⁸ | 0.014 | 1.3 × 10⁻⁹ | 0.86 | 2.4 × 10⁻¹⁵ | 0.86 |
| | | | | | | | | | (0.82-0.90) | | (0.84-0.90) |
| rs5167 | 19 | 50.1 | APOC2 | Exon | 0.35 | 3.4 × 10⁻⁸ | | | | | |

Chr = Chromosome; MB = position in megabases; MAF = minor allele frequency in controls; OR = odds ratio for the minor allele; 95% CI = 95% confidence interval; UTR = untranslated region.
* rs2075650 is in linkage disequilibrium with rs429358, the APOE ε4 SNP (r² = 0.48).
† This SNP was genotyped in a subsample of 3333 cases and 6460 controls.

TABLE 2
SNPs showing association with AD at P ≦ 1 × 10⁻⁵
(excluding SNPs at the APOE, CLU and PICALM loci).

| | | | Closest | Location Relative | GWAS | | |
|---|---|---|---|---|---|---|---|
| SNP | Chr | MB | RefSeq Gene | to Gene | P-value | OR | 95% CI |
| rs11894266 | 2 | 170.3 | SSB | 5′ | 6.9 × 10⁻⁷ | 0.86 | 0.81-0.91 |
| rs610932 | 11 | 59.7 | MS4A6A | 3′UTR | 1.4 × 10⁻⁶ | 0.87 | 0.82-0.92 |
| rs10501927 | 11 | 99.3 | CNTN5 | Intronic | 2.0 × 10⁻⁶ | 1.18 | 1.10-1.26 |
| rs9446432 | 6 | 72.4 | | Intergenic | 2.8 × 10⁻⁶ | 1.28 | 1.15-1.41 |
| rs7561528 | 2 | 127.6 | BIN1 | 5′ | 3.0 × 10⁻⁶ | 1.16 | 1.09-1.24 |
| rs744373 | 2 | 127.6 | BIN1 | 5′ | 3.2 × 10⁻⁶ | 1.17 | 1.09-1.25 |
| rs662196 | 11 | 59.7 | MS4A6A | Intronic | 5.2 × 10⁻⁶ | 0.88 | 0.83-0.93 |
| rs583791 | 11 | 59.7 | MS4A6A | Intronic | 5.3 × 10⁻⁶ | 0.88 | 0.83-0.93 |
| rs676309 | 11 | 59.8 | MS4A4E | 5′ | 6.3 × 10⁻⁶ | 1.14 | 1.08-1.20 |
| rs1157242 | 8 | 37.2 | | Intergenic | 7.0 × 10⁻⁶ | 1.17 | 1.10-1.26 |
| rs1539053 | 1 | 57.9 | DAB1 | Intronic | 7.1 × 10⁻⁶ | 0.88 | 0.83-0.93 |
| rs11827375 | 11 | 76.0 | C11orf30 | 3′ | 7.2 × 10⁻⁶ | 1.23 | 1.12-1.35 |
| rs1408077 | 1 | 205.9 | CR1 | Intronic | 8.3 × 10⁻⁶ | 1.17 | 1.09-1.25 |
| rs9384428 | 6 | 156.5 | | Intergenic | 8.5 × 10⁻⁶ | 1.14 | 1.08-1.21 |
| rs6701713 | 1 | 205.9 | CR1 | Intronic | 8.7 × 10⁻⁶ | 1.17 | 1.09-1.25 |
| rs3818361 | 1 | 205.9 | CR1 | Intronic | 9.2 × 10⁻⁶ | 1.17 | 1.09-1.25 |

Chr = Chromosome; MB = position in megabases; OR = odds ratio for the minor allele; 95% CI = 95% confidence interval; UTR = untranslated region.

TABLE 3
Summary statistics for SNPs selected from meta-analysis of stage1, ADNI,
TGEN and EADI1 datasets and genotyped in the full stage 2 sample.

| | | | | | | Meta-analysis Stage1&2, | |
|---|---|---|---|---|---|---|---|
| | | | | Follow-up Stage 2: | | EADI1, ADNI, TGEN, | |
| | | | Discovery Stage1, | 3,262 cases, | | CHARGE P^{b}: 11607 cases, | |
| | | | EADI1, ADNI & | 5064 controls | | 31871 controls | |
| | | Closet | TGEN P^{a}: 6978 cases, | | Odds Ratio | | Odds Ratio |
| SNP | Chr | gene | 13903 controls | P | (95% CI) | P | (95% CI) |
| rs3818361 | 1 | CR1 | 5.5 × 10^{-12d} | 8.9 × 10⁻³ | 1.13 (1.03-1.25) | 1.9 × 10⁻¹² | 1.17 (1.12-1.22) |
| rs744373 | 2 | BIN1 | 2.0 × 10^{-8d} | 6.5 × 10⁻³ | 1.12 (1.03-1.21) | 2.1 × 10⁻¹² | 1.14 (1.10-1.19) |
| rs10501927 | 11 | CNTN5 | 1.6 × 10⁻⁷ | 9.9 × 10⁻¹ | 1.00 (0.92-1.09) | | |
| rs1858973 | 16 | IQCK | 4.9 × 10⁻⁷ | 5.2 × 10⁻¹ | 0.97 (0.88-1.07) | | |
| rs10761558 | 10 | CDC2 | 1.3 × 10⁻⁶ | 9.2 × 10⁻¹ | 1.00 (0.93-1.09) | | |
| rs4782279 | 16 | IQCK | 1.3 × 10⁻⁶ | 4.0 × 10⁻¹ | 0.96 (0.87-1.06) | | |
| rs610932 | 11 | MS4A4E | 1.5 × 10^{-6d} | 2.8 × 10⁻² | 0.92 (0.85-0.99) | 1.1 × 10⁻⁷ | 0.91 (0.88-0.94) |
| rs3764650 | 19 | ABCA7 | 1.5 × 10^{-6d} | 1.3 × 10⁻⁴ | 1.27 (1.12-1.43) | 7.3 × 10⁻¹⁰ | 1.22 (1.14-1.30) |
| rs4958112 | 5 | FSTL4 | 1.6 × 10⁻⁶ | 7.2 × 10⁻¹ | 1.01 (0.94-1.09) | | |
| rs7191155 | 16 | IQCK | 2.2 × 10⁻⁶ | 4.2 × 10⁻¹ | 0.96 (0.87-1.06) | | |
| rs8055533 | 16 | CLEC16A | 2.8 × 10⁻⁶ | 9.5 × 10⁻¹ | 1.00 (0.93-1.07) | | |
| rs739565 | 16 | IQCK | 3.3 × 10⁻⁶ | 7.2 × 10⁻¹ | 1.01 (0.94-1.09) | | |
| rs3135344 | 6 | BTNL2 | 5.2 × 10⁻⁶ | 2.3 × 10⁻¹ | 1.07 (0.96-1.19) | | |
| rs3809278 | 12 | CUX2 | 5.3 × 10⁻⁶ | 9.1 × 10⁻¹ | 1.01 (0.90-1.13) | | |
| rs670139 | 11 | MS4A6A | 5.6 × 10^{-6d} | 9.2 × 10⁻⁴ | 1.14 (1.06-1.23) | 1.2 × 10⁻⁹ | 1.12 (1.08-1.16) |
| rs11894266 | 2 | SSB | 5.9 × 10⁻⁶ | 0.6821³ | 0.99 (0.92-1.06) | | |
| rs1539053 | 1 | DAB1 | 6.7 × 10⁻⁶ | 2.6 × 10⁻¹ | 1.05 (0.97-1.13) | | |
| rs11767557 | 7 | EPHA1 | 7.2 × 10⁻⁶ | 8.4 × 10⁻¹ | 0.99 (0.90-1.09) | | |
| rs9314866 | 9 | GNAQ | 8.2 × 10⁻⁶ | 4.2 × 10⁻¹ | 0.97 (0.89-1.05) | | |
| rs7573507 | 2 | ARHGAP25 | 8.5 × 10⁻⁶ | 6.8 × 10⁻¹ | 0.96 (0.79-1.17) | | |
| rs4571225 | 3 | IL1RAP | 9.9 × 10⁻⁶ | 8.1 × 10⁻¹ | 0.98 (0.79-1.20) | | |

Chr, chromosome;
95% CI, 95% confidence interval
^{a}P values were obtained by performing an inverse-variance weighted meta-analysis of the Stage 1, ADNI and TGEN, and combining the resultant P-values with those from the EADI1 study using Fisher's combined probability test.
^{b}Inverse variance weighted meta-analysis
^{c} rs11894266 failed to optimize, rs13010581 genotyped as proxy (r2 = 1, D′ = 1),
^{d}P-values based on inverse variance weighted meta-analysis of Stage 1&2, ADNI, TGEN and EADI1 for SNPs rs3764650, rs670139, rs744373, rs610932 & rs3818361were 2.8 × 10−7, 2.9 × 10−6, 3.6 × 10−9, 3.8 × 10−7 and 2.5 × 10−12, respectively.

TABLE S1
Sample size and descriptive statistics for the discovery sample.

| | | | | | UCL: | UCL: | | |
|---|---|---|---|---|---|---|---|---|
| | TOTAL | MRC | ART | WASHU | PRION | LASER | NIMH | BONN |
| | Geographical Region | | | | | | | |
| | | UK/Ire | UK | USA | UK | UK | USA | Germany |
| Illumina Chip | | 610 | 610 | 610 | 610 | 610 | 610 | 610 |
| AD Cases | | | | | | | | |
| n, total | 4957 | 1221 | 1223 | 503 | 278 | 53 | 155 | 680 |
| n, passed QC | 3941 | 1009 | 960 | 424 | 211 | 47 | 127 | 555 |
| % Female | 62.7 | 70.4 | 60.4 | 56.1 | 58.8 | 74.5 | 63.0 | 63.9 |
| % Neuropathological | 6.6 | 0.0 | 8.3 | 0.0 | 0.0 | 0.0 | 0.0 | 0.0 |
| Confirmed | | | | | | | | |
| Mean Age at onset | 73.2 | 75.7 | 72.1^{‡} | 73.1 | 63.2^{‡} | N/A | 72.1 | 70.5 |
| Age at assessment, mean | 78.6 | 80.9 | 78.4 | 80.5 | N/A | 80.6 | 81.3 | 72.9 |
| Age at death, mean * | 80.4 | N/A | 82.9 | 84.1 | N/A | N/A | N/A | N/A |
| Elderly Screened Controls | | | | | | | | |
| n, total | 2857 | 1044 | 121 | 300 | — | — | — | 137 |
| n, passed QC | 2078 | 873 | 82 | 233 | — | — | — | 37 |
| % Female | 58.0 | 62.0 | 59.8 | 66.1 | — | — | — | 64.9 |
| % Neuropathological | 8.3 | 0.0 | 23.2 | 0.0 | — | — | — | 0.0 |
| Confirmed | | | | | | | | |
| Age at assessment, mean | 75.2 | 75.9 | 76.7 | 77.7 | — | — | — | 79.5 |
| Age at death, mean * | 80.4 | N/A | 81.6 | N/A | — | — | — | N/A |
| Population Controls | | | | | | | | |
| n, total | 6825 | — | — | — | — | — | — | — |
| n, passed QC | 5770 | — | — | — | — | — | — | — |
| % Female | 51.8 | — | — | — | — | — | — | — |
| % Neuropathological | 0.0 | — | — | — | — | — | — | — |
| Confirmed | | | | | | | | |
| Age at assessment, mean | 48.6 | — | — | — | — | — | — | — |
| Age at death, mean * | N/A | — | — | — | — | — | — | — |

| | MAYO | 1958BC | CORIELL | KORA | HNR | ALS |
|---|---|---|---|---|---|---|
| | Geographical Region | | | | | |
| | USA | UK | USA | Germany | Germany | UK/USA |
| Illumina Chip | 300 | 550 | 550 | 550 | 550 | 300 |
| AD Cases | | | | | | |
| n, total | 844 | — | — | — | — | — |
| n, passed QC | 608 | — | — | — | — | — |
| % Female | 57.4 | — | — | — | — | — |
| % Neuropathological | 29.6 | — | — | — | — | — |
| Confirmed | | | | | | |
| Mean Age at onset | 74.1^{‡} | — | — | — | — | — |
| Age at assessment, mean | N/A | — | — | — | — | — |
| Age at death, mean * | 73.9^{†} | — | — | — | — | — |
| Elderly Screened Controls | | | | | | |
| n, total | 1255 | — | — | — | — | — |
| n, passed QC | 853 | — | — | — | — | — |
| % Female | 51.2 | — | — | — | — | — |
| % Neuropathological | 17.9 | — | — | — | — | — |
| Confirmed | | | | | | |
| Age at assessment, mean | 73.6 | — | — | — | — | — |
| Age at death, mean * | 71.5 | — | — | — | — | — |
| Population Controls | | | | | | |
| n, total | — | 4032 | 808 | 481 | 380 | 1124 |
| n, passed QC | — | 3751 | 697 | 434 | 353 | 535 |
| % Female | — | 50.8 | 59.1 | 49.1 | 53.0 | 50.3 |
| % Neuropathological | — | 0.0 | 0.0 | 0.0 | 0.0 | 0.0 |
| Confirmed | | | | | | |
| Age at assessment, mean | — | 44.0 | 58.1 | 56.0 | 54.6 | 57.2 |
| Age at death, mean * | — | N/A | N/A | N/A | N/A | N/A |

* Only available for neuropathological samples
^{†}Mean age at death for autopsy confirmed samples only (n = 246). Age at onset data is not available for these participants.
^{‡}Age at onset only available for a proportion of the sample
Ire = Republic of Ireland;
610 = Illumina 610-quad;
550 = Illumina HumanHap550;
300 = Illumina HumanHap300

TABLE S2
Sample size and descriptive statistics for the original stage 2 sample.

| | TOTAL | BELGIUM | MRC | ART | BONN | GREEK |
|---|---|---|---|---|---|---|
| | Geographical Region | | | | | |
| | | Belgium | UK/Ire | UK | Germany | Greece |
| AD Cases | | | | | | |
| n | 2023 | 1091 | 198 | 82 | 248 | 404 |
| % Female | 66.2 | 66.2 | 64.6 | 79.3 | 65.2 | 64.6 |
| % Neuropathological | 0.0 | 7.5 | 0.0 | 0.0 | 0.0 | 0.0 |
| Confirmed | | | | | | |
| Mean Age at onset | 73.2 | 74.4 | 76.2 | 73.7^{‡} | 69.4^{‡} | 69.0^{‡} |
| Age at assessment, mean | 78.2 | 78.6 | 81.7 | 78.0 | 75.7 | 76.7 |
| Age at death, mean * | N/A | N/A | N/A | N/A | N/A | N/A |
| Elderly Screened Controls | | | | | | |
| n | 2340 | 662 | 372 | 305 | 618 | 383^{†} |
| % Female | 59.1% | 58.4% | 64.2% | 67.7% | 65.5% | 37.7% |
| % Neuropathological | 0.0% | 0.0% | 0.0% | 0.0% | 0.0% | 0.0% |
| Confirmed | | | | | | |
| Age at assessment, mean | 69.8 | 63.0 | 76.6 | 74.0 | 79.6 | 54.9 |
| Age at death, mean * | N/A | N/A | N/A | N/A | N/A | N/A |

* Only available for neuropathological samples
^{†} 171 aged-matched screened controls, 212 population controls
^{‡}Age at onset only available for a proportion of the sample

TABLE S3
Descriptive statistics for full Stage 2 sample.

| | Stage2 Sample | | | | | | | | | |
|---|---|---|---|---|---|---|---|---|---|---|
| | Total stage2 | | | | | | UCL- | | | |
| | Sample | MRC | ART | Belgium | Bonn | Caerphilly | PRION | Laser | Greece | Munich |
| Genotyping Platform | Sequenom | | | | | | | | | |
| AD Cases | | | | | | | | | | |
| n | 3262 | 291 | 628 | 1078 | 347 | 52 | 92 | 42 | 404 | 328 |
| % Female | 64.4 | 63.5 | 61.3 | 66.2 | 79.3 | 0 | 57.1 | 69.0 | 64.6 | 66.8 |
| Age at onset, Mean | 72.9 | 75.7 | 70.6‡ | 74.9 | 70.3 | N/A | 61.2 | N/A | 69.0‡ | 70.5 |
| Age at Interview, Mean | 77.7 | 81.1 | 78.4† | 78.6 | 76.2 | N/A | N/A | 79.3 | 76.7 | 73.2 |
| Age at death, Mean | 81.6 | N/A | 81.6† | N/A | N/A | N/A | N/A | N/A | N/A | N/A |
| Controls | | | | | | | | | | |
| n | 5064 | 451 | 399 | 906 | 896 | 0 | 0 | 0 | 364 | 2048 |
| % Female | 56.2 | 62.0 | 60.5 | 58.4 | 68.0 | N/A | N/A | N/A | 37.2 | 51.2 |
| Age at Interview, Mean | 62.4 | 76.5 | 74.0† | 63.0 | 79.6 | N/A | N/A | N/A | 54.2 | 50.7 |
| Age at death, Mean | 76.7 | N/A | 76.7† | N/A | N/A | N/A | N/A | N/A | N/A | N/A |
| Incidence studies | | | | | | | | | | |
| Cohort at risk | | | | | | | | | | |
| % Female | | | | | | | | | | |
| Age at start | | | | | | | | | | |
| Incident AD cases | | | | | | | | | | |

†Age at interview not available for 438 AD cases and 104 controls. Age at death is provided for these subjects where available.
‡Age at onset data only available for less than 75% of the sample

TABLE S4
Quality control filters applied to individuals in each cohort.

| | | | Missing | | X-Chr | X-Chr |
|---|---|---|---|---|---|---|
| | | Illumina | Genotype | Autosomal | Heterozygosity: | Heterozygosity: |
| Cohort | N | Chip | Rate | Heterozygosity | Males | Females |
| 610 | 5715 | 610 | ≦0.01 | 0.325-0.337 | <0.02 | 0.25-0.4 |
| Mayo | 2099 | 300 | ≦0.01 | 0.3455-0.356 | <0.015 | 0.31-0.385 |
| 1958BC | 2596 | 550 | ≦0.01 | 0.33-0.34 | <0.007 | 0.294-0.37 |
| (T1DGC) | | | | | | |
| 1958BC | 1436 | 550 | ≦0.01 | 0.329-0.34 | <0.011 | 0.295-0.38 |
| (Sanger) | | | | | | |
| Coriell | 808 | 550 | ≦0.01 | 0.345-0.356 | <0.015 | 0.32-0.39 |
| ALS | 1124 | 300 | ≦0.01 | 0.345-0.355 | <0.015 | 0.32-0.395 |
| Controls | | | | | | |
| HNR | 380 | 550 | ≦0.01 | 0.33-0.34 | <0.005 | 0.30-0.37 |
| Kora | 481 | 550 | ≦0.01 | 0.33-0.34 | <0.02 | 0.30-0.37 |

N = number of individuals;
Chr = chromosome.

TABLE S5
Breakdown of individuals excluded by group.

| | Number of | Missing | | | | | Non- | | Number of |
|---|---|---|---|---|---|---|---|---|---|
| | Individuals | Genotype | Auto | X-Chr | Gender | Related- | European | Genetic | Individuals |
| Group | before QC | Rate | Het | Het | Check ^{a} | ness | Ancestry | Outlier ^{b} | after QC |
| 610 | 5715 | 679 | 140 | 5 | 71 | 168 | 11 | 83 | 4558 |
| Mayo | 2099 | 426 | 151 | 1 | 0 | 19 | 4 | 37 | 1461 |
| 1958BC | 2596 | 57 | 2 | 2 | 1 ^{a} | 4 | 0 | 12 | 2519 |
| (T1DGC) | | | | | | | | | |
| 1958BC | 1436 | 87 | 108 | 4 | 0 | 1 | 0 | 4 | 1232 |
| (Sanger) | | | | | | | | | |
| Coriell | 808 | 49 | 21 | 8 | 2 ^{a} | 0 | 1 | 32 | 697 |
| ALS | 1124 | 118 | 139 | 0 | 0 | 313 | 0 | 19 | 535 |
| Controls | | | | | | | | | |
| HNR | 380 | 22 | 4 | 1 | 0 | 0 | 0 | 0 | 353 |
| Kora | 481 | 31 | 13 | 1 | 0 | 1 | 0 | 1 | 434 |
| Total | 14639 | 1469 | 578 | 22 | 71 | 506 | 16 | 188 | 11789 |

Auto Het = mean autosomal heterozygosity.
X-Chr Het = mean X chromosome heterozygosity.
Note that 311 individuals were included in both the Coriell and ALS control cohort.
^{a} Population controls were not excluded if there was a discrepancy between reported gender and genotype-determined gender.
^{b} Outliers identified by EIGENSTRAT.

TABLE S6
Summary of the numbers of SNPs before and after quality control.

| | | Autosomal | Autosomal | | |
|---|---|---|---|---|---|
| SNP | | SNPs | SNPs | | |
| Category | Chip | before QC | after QC | Cases | Controls |
| 1 | 610 + 550 + 300 | 266714 | 257253 | 3,941 | 7,848 |
| 2 | 610 + 550 | 202516 | 199196 | 3,333 | 6,460 |
| 3 | 610 + 300 | 7744 | 7196 | 3,941 | 2,613 |
| 4 | 610 | 105614 | 65560 | 3,333 | 1,225 |
| | | 582588 | 529205 | | |

TABLE S7
The effect of including principal components (PCs) extracted from
EIGENSTRAT on the genomic control inflation factor, λ. These values
are based on analysis of SNPs common to the IIlumina 610-quad,
HumanHap550 and HumanHap300 chips (SNP category 1).

| Principal Components | |
|---|---|
| included | λ |
| 0 | 1.079 |
| 1 | 1.047 |
| 1-2 | 1.042 |
| 1-3 | 1.038 |
| 1-4 | 1.038 |
| 1-5 | 1.038 |
| 1-6 | 1.039 |
| 1-7 | 1.038 |
| 1-8 | 1.038 |
| 1-9 | 1.039 |
| 1-10 | 1.040 |

TABLE S8
Significance thresholds (χ² values) employed to exclude SNPs showing
inter-chip or inter-cohort differences. SNPs with χ² values
exceeding these thresholds were excluded.

| | 1 | 2 | 3 | 4 |
|---|---|---|---|---|
| Controls compared | 610 + 550 + 300 | 610 + 550 | 610 + 300 | 610 |
| HNR/Kora | 15.0 | 12.0 | N/A | N/A |
| 610 (German)/HNR + Kora | 17.5 | 10.0 | N/A | N/A |
| 1958BC (Sanger + T1DGC)/ALS (UK) | 6.0 | N/A | N/A | N/A |
| 1958BC (Sanger)/1958BC (T1DGC) | 11.5 | 10.0 | N/A | N/A |
| 610 (UK)/ALS (UK) | 6.0 | N/A | 13.0 | N/A |
| 1958BC (Sanger + T1DGC)/610 (UK) | 11.0 | 11.0 | N/A | N/A |
| Mayo/ALS (US) | 10.0 | N/A | 5.0 | N/A |
| Coriell/Mayo + ALS (US) | 25.0 | N/A | N/A | N/A |
| 610 (US)/Mayo + ALS (US) | 25.0 | N/A | 14.0 | N/A |
| 610 (US)/Coriell | 20.0 | 18.5 | N/A | N/A |

N/A = not applicable.

TABLE S9
SNPs showing association with AD (P ≦ 1 × 10⁻⁴) in the GWAS.

| | | Position | Minor | Number of | | | |
|---|---|---|---|---|---|---|---|
| SNP | Chr | (basepairs) | Allele | Individuals | OR | 95% CI | P-value |
| rs2075650 | 19 | 50087459 | C | 11784 | 2.53 | (2.36-2.71) | 1.8 × 10⁻¹⁵⁷ |
| rs157580 | 19 | 50087106 | G | 11784 | 0.63 | (0.59-0.66) | 9.6 × 10⁻⁵⁴ |
| rs6859 | 19 | 50073874 | T | 11766 | 1.46 | (1.37-1.54) | 6.9 × 10⁻⁴¹ |
| rs8106922 | 19 | 50093506 | G | 11771 | 0.68 | (0.64-0.71) | 5.4 × 10⁻³⁹ |
| rs405509 | 19 | 50100676 | C | 11788 | 0.70 | (0.66-0.73) | 4.9 × 10⁻³⁷ |
| rs10402271 | 19 | 50021054 | G | 11787 | 1.36 | (1.28-1.43) | 1.5 × 10⁻²⁶ |
| rs439401 | 19 | 50106291 | T | 9773 | 0.72 | (0.67-0.76) | 2.7 × 10⁻²³ |
| rs2927438 | 19 | 49933947 | T | 11774 | 1.25 | (1.16-1.32) | 3.0 × 10⁻¹¹ |
| rs377702 | 19 | 50054507 | T | 11779 | 1.20 | (1.13-1.27) | 8.4 × 10⁻¹¹ |
| rs11136000 | 8 | 27520436 | A | 11756 | 0.84 | (0.79-0.88) | 1.4 × 10⁻⁹ |
| rs1048699 | 19 | 50342226 | A | 11774 | 1.32 | (1.20-1.43) | 1.5 × 10⁻⁹ |
| rs1114832 | 19 | 50328041 | A | 11785 | 1.31 | (1.19-1.42) | 2.2 × 10⁻⁹ |
| rs1871047 | 19 | 50043586 | G | 11784 | 0.85 | (0.80-0.89) | 1.3 × 10⁻⁸ |
| rs3851179 | 11 | 85546288 | A | 11789 | 0.85 | (0.80-0.89) | 1.9 × 10⁻⁸ |
| rs5167 | 19 | 50140305 | C | 11789 | 1.17 | (1.10-1.24) | 3.4 × 10⁻⁸ |
| rs2582367 | 8 | 27535944 | G | 9701 | 0.85 | (0.80-0.90) | 2.1 × 10⁻⁷ |
| rs7941541 | 11 | 85536186 | C | 9791 | 0.84 | (0.78-0.89) | 2.1 × 10⁻⁷ |
| rs8103315 | 19 | 49946008 | T | 9788 | 1.24 | (1.14-1.35) | 2.9 × 10⁻⁷ |
| rs3760627 | 19 | 50149020 | G | 11754 | 1.15 | (1.09-1.21) | 4.0 × 10⁻⁷ |
| rs2965101 | 19 | 49929652 | C | 11789 | 0.86 | (0.80-0.90) | 4.5 × 10⁻⁷ |
| rs11894266 | 2 | 170344888 | C | 9793 | 0.86 | (0.80-0.91) | 6.8 × 10⁻⁷ |
| rs543293 | 11 | 85497725 | A | 11787 | 0.86 | (0.81-0.91) | 6.9 × 10⁻⁷ |
| rs12610605 | 19 | 50062678 | T | 9793 | 0.81 | (0.74-0.88) | 7.7 × 10⁻⁷ |
| rs1237999 | 11 | 85492678 | C | 11788 | 0.87 | (0.81-0.91) | 1.1 × 10⁻⁶ |
| rs610932 | 11 | 59695883 | A | 11779 | 0.87 | (0.82-0.92) | 1.4 × 10⁻⁶ |
| rs10501927 | 11 | 99262939 | G | 11786 | 1.18 | (1.10-1.26) | 2.0 × 10⁻⁶ |
| rs541458 | 11 | 85465999 | C | 11789 | 0.87 | (0.81-0.91) | 2.3 × 10⁻⁶ |
| rs9446432 | 6 | 72413481 | C | 9783 | 1.28 | (1.15-1.41) | 2.8 × 10⁻⁶ |
| rs7561528 | 2 | 127606107 | T | 9779 | 1.16 | (1.09-1.23) | 3.0 × 10⁻⁶ |
| rs744373 | 2 | 127611085 | G | 9783 | 1.17 | (1.09-1.24) | 3.2 × 10⁻⁶ |
| rs536841 | 11 | 85465472 | C | 9788 | 0.86 | (0.80-0.91) | 3.5 × 10⁻⁶ |
| rs659023 | 11 | 85502507 | T | 9759 | 0.86 | (0.81-0.91) | 3.6 × 10⁻⁶ |
| rs662196 | 11 | 59699333 | G | 11783 | 0.88 | (0.83-0.92) | 5.1 × 10⁻⁶ |
| rs583791 | 11 | 59703828 | C | 11759 | 0.88 | (0.83-0.92) | 5.3 × 10⁻⁶ |
| rs440277 | 19 | 50053064 | A | 9793 | 0.86 | (0.80-0.91) | 5.4 × 10⁻⁶ |
| rs8111069 | 19 | 50175278 | G | 9768 | 1.16 | (1.08-1.23) | 6.1 × 10⁻⁶ |
| rs676309 | 11 | 59758149 | C | 11785 | 1.14 | (1.07-1.20) | 6.3 × 10⁻⁶ |
| rs1157242 | 8 | 37158523 | T | 11787 | 1.17 | (1.09-1.25) | 7.0 × 10⁻⁶ |
| rs1539053 | 1 | 57872295 | T | 11788 | 0.88 | (0.83-0.93) | 7.1 × 10⁻⁶ |
| rs11827375 | 11 | 75985920 | T | 9775 | 1.23 | (1.12-1.34) | 7.2 × 10⁻⁶ |
| rs1408077 | 1 | 205870764 | T | 11705 | 1.17 | (1.09-1.25) | 8.3 × 10⁻⁶ |
| rs9384428 | 6 | 156541223 | G | 11782 | 1.14 | (1.07-1.21) | 8.5 × 10⁻⁶ |
| rs6701713 | 1 | 205852912 | T | 11786 | 1.17 | (1.09-1.24) | 8.7 × 10⁻⁶ |
| rs3818361 | 1 | 205851591 | A | 11787 | 1.17 | (1.09-1.24) | 9.2 × 10⁻⁶ |
| rs1562990 | 11 | 59779663 | C | 11786 | 0.88 | (0.83-0.93) | 1.0 × 10⁻⁵ |
| rs7933349 | 11 | 99363242 | G | 11785 | 1.15 | (1.07-1.21) | 1.1 × 10⁻⁵ |
| rs1994313 | 4 | 175953201 | T | 11772 | 0.88 | (0.83-0.93) | 1.2 × 10⁻⁵ |
| rs618679 | 11 | 85349350 | T | 9775 | 0.84 | (0.78-0.91) | 1.2 × 10⁻⁵ |
| rs4571225 | 3 | 191807148 | G | 4557 | 0.60 | (0.47-0.75) | 1.3 × 10⁻⁵ |
| rs569214 | 8 | 27543709 | T | 11788 | 0.88 | (0.83-0.93) | 1.3 × 10⁻⁵ |
| rs1457850 | 15 | 93332855 | G | 9755 | 0.87 | (0.82-0.92) | 1.4 × 10⁻⁵ |
| rs3764650 | 19 | 997520 | C | 9790 | 1.25 | (1.12-1.37) | 1.6 × 10⁻⁵ |
| rs677909 | 11 | 85435237 | G | 11751 | 0.88 | (0.82-0.93) | 1.6 × 10⁻⁵ |
| rs10761558 | 10 | 62193476 | T | 4549 | 1.26 | (1.13-1.39) | 1.6 × 10⁻⁵ |
| rs4705563 | 5 | 113007438 | G | 4555 | 0.81 | (0.73-0.89) | 1.7 × 10⁻⁵ |
| rs10899221 | 11 | 75859279 | T | 9787 | 1.27 | (1.13-1.41) | 1.8 × 10⁻⁵ |
| rs10425074 | 19 | 50331964 | C | 11778 | 1.15 | (1.07-1.22) | 1.9 × 10⁻⁵ |
| rs11667640 | 19 | 50071631 | T | 11782 | 0.77 | (0.68-0.87) | 2.3 × 10⁻⁵ |
| rs667897 | 11 | 59693555 | C | 11777 | 0.89 | (0.84-0.93) | 2.5 × 10⁻⁵ |
| rs387976 | 19 | 50070900 | C | 9793 | 0.87 | (0.82-0.93) | 2.7 × 10⁻⁵ |
| rs12781740 | 10 | 64047973 | T | 9789 | 0.83 | (0.76-0.90) | 2.7 × 10⁻⁵ |
| rs4803750 | 19 | 49939467 | G | 9790 | 0.77 | (0.67-0.86) | 2.8 × 10⁻⁵ |
| rs272610 | 8 | 81644892 | G | 11781 | 0.89 | (0.83-0.93) | 2.9 × 10⁻⁵ |
| rs9876068 | 3 | 142901893 | C | 4557 | 0.62 | (0.49-0.77) | 2.9 × 10⁻⁵ |
| rs7679849 | 4 | 122616456 | A | 11787 | 1.18 | (1.09-1.27) | 3.1 × 10⁻⁵ |
| rs10898438 | 11 | 85535901 | C | 9780 | 0.88 | (0.83-0.93) | 3.3 × 10⁻⁵ |
| rs13071397 | 3 | 20805715 | T | 9719 | 1.23 | (1.11-1.36) | 3.8 × 10⁻⁵ |
| rs11932698 | 4 | 122607113 | A | 11783 | 1.18 | (1.08-1.26) | 3.8 × 10⁻⁵ |
| rs12449868 | 17 | 69675770 | G | 9784 | 0.88 | (0.82-0.93) | 4.0 × 10⁻⁵ |
| rs1957325 | 14 | 84965914 | A | 11777 | 0.80 | (0.72-0.89) | 4.0 × 10⁻⁵ |
| rs4789626 | 17 | 69679180 | T | 11783 | 0.89 | (0.84-0.94) | 4.3 × 10⁻⁵ |
| rs6716044 | 2 | 104554436 | A | 11784 | 0.81 | (0.73-0.89) | 4.8 × 10⁻⁵ |
| rs2077815 | 11 | 85350031 | G | 11785 | 0.87 | (0.80-0.92) | 4.9 × 10⁻⁵ |
| rs7715371 | 5 | 148659187 | T | 9777 | 0.80 | (0.71-0.88) | 5.0 × 10⁻⁵ |
| rs12692925 | 2 | 170340174 | C | 9791 | 0.88 | (0.82-0.93) | 5.1 × 10⁻⁵ |
| rs276968 | 16 | 84789677 | G | 11780 | 0.86 | (0.80-0.92) | 5.1 × 10⁻⁵ |
| rs597668 | 19 | 50400728 | C | 11787 | 1.16 | (1.08-1.24) | 5.2 × 10⁻⁵ |
| rs2448166 | 8 | 98523312 | G | 11785 | 1.21 | (1.10-1.32) | 5.3 × 10⁻⁵ |
| rs12686004 | 9 | 106693247 | A | 9778 | 1.21 | (1.10-1.32) | 5.3 × 10⁻⁵ |
| rs10474519 | 5 | 76929703 | T | 9781 | 1.23 | (1.11-1.35) | 5.4 × 10⁻⁵ |
| rs1328179 | 1 | 192019846 | T | 11763 | 0.89 | (0.84-0.94) | 5.6 × 10⁻⁵ |
| rs1474198 | 4 | 72987876 | C | 11785 | 0.89 | (0.84-0.94) | 5.7 × 10⁻⁵ |
| rs540170 | 11 | 59636614 | T | 9793 | 0.88 | (0.83-0.93) | 5.7 × 10⁻⁵ |
| rs9539818 | 13 | 63054607 | G | 9763 | 0.81 | (0.72-0.89) | 6.0 × 10⁻⁵ |
| rs581133 | 11 | 59638882 | C | 9790 | 0.88 | (0.83-0.93) | 6.2 × 10⁻⁵ |
| rs17005633 | 4 | 83540832 | T | 9761 | 1.18 | (1.08-1.27) | 6.3 × 10⁻⁵ |
| rs2767576 | 6 | 156921465 | C | 4554 | 1.30 | (1.14-1.48) | 6.3 × 10⁻⁵ |
| rs527162 | 11 | 85393384 | G | 11789 | 0.87 | (0.80-0.92) | 6.3 × 10⁻⁵ |
| rs12652626 | 5 | 134511487 | T | 6548 | 1.61 | (1.27-2.02) | 6.4 × 10⁻⁵ |
| rs10883543 | 10 | 102542742 | C | 11782 | 1.19 | (1.09-1.29) | 6.5 × 10⁻⁵ |
| rs12671881 | 7 | 153812564 | G | 11775 | 0.89 | (0.84-0.94) | 6.6 × 10⁻⁵ |
| rs10063333 | 5 | 20623020 | G | 11753 | 1.12 | (1.05-1.18) | 6.6 × 10⁻⁵ |
| rs17642472 | 5 | 148663280 | T | 9791 | 0.78 | (0.69-0.88) | 6.8 × 10⁻⁵ |
| rs12697730 | 5 | 82944230 | A | 9792 | 1.14 | (1.06-1.21) | 6.8 × 10⁻⁵ |
| rs10517459 | 4 | 37636321 | C | 11773 | 1.20 | (1.09-1.3) | 6.8 × 10⁻⁵ |
| rs4381764 | 2 | 201835108 | C | 4557 | 0.61 | (0.47-0.77) | 6.8 × 10⁻⁵ |
| rs11606287 | 11 | 47364015 | T | 9677 | 1.13 | (1.06-1.2) | 7.3 × 10⁻⁵ |
| rs6467136 | 7 | 126952194 | A | 9719 | 1.13 | (1.06-1.19) | 7.3 × 10⁻⁵ |
| rs1917899 | 11 | 36068192 | C | 9790 | 0.88 | (0.82-0.93) | 7.3 × 10⁻⁵ |
| rs7255066 | 19 | 49837943 | C | 11778 | 0.88 | (0.82-0.93) | 7.4 × 10⁻⁵ |
| rs17098701 | 5 | 141836893 | G | 4558 | 0.64 | (0.51-0.79) | 7.4 × 10⁻⁵ |
| rs309568 | 5 | 82933474 | G | 9792 | 1.14 | (1.06-1.21) | 7.5 × 10⁻⁵ |
| rs1983659 | 22 | 26252759 | G | 11787 | 0.89 | (0.84-0.94) | 7.7 × 10⁻⁵ |
| rs7257916 | 19 | 50174724 | G | 9791 | 0.89 | (0.83-0.94) | 7.8 × 10⁻⁵ |
| rs986251 | 5 | 20606744 | T | 11786 | 1.12 | (1.05-1.18) | 7.9 × 10⁻⁵ |
| rs7926344 | 11 | 59718742 | A | 9757 | 0.88 | (0.83-0.93) | 7.9 × 10⁻⁵ |
| rs7017417 | 8 | 27401222 | C | 11787 | 1.18 | (1.08-1.27) | 8.0 × 10⁻⁵ |
| rs10496105 | 2 | 64174250 | T | 11789 | 1.18 | (1.08-1.28) | 8.1 × 10⁻⁵ |
| rs1341316 | 1 | 57860377 | C | 11784 | 0.89 | (0.83-0.94) | 8.2 × 10⁻⁵ |
| rs7929589 | 11 | 59731654 | A | 9791 | 0.88 | (0.83-0.93) | 8.3 × 10⁻⁵ |
| rs17296283 | 16 | 68143729 | A | 9788 | 1.27 | (1.12-1.42) | 8.5 × 10⁻⁵ |
| rs6785659 | 3 | 5817729 | A | 9791 | 1.13 | (1.06-1.19) | 8.7 × 10⁻⁵ |
| rs836326 | 4 | 173387832 | T | 11783 | 1.12 | (1.05-1.18) | 8.8 × 10⁻⁵ |
| rs4720922 | 7 | 1699628 | T | 11755 | 1.12 | (1.06-1.19) | 9.3 × 10⁻⁵ |
| rs1241486 | 14 | 24667999 | G | 11775 | 0.90 | (0.84-0.94) | 9.5 × 10⁻⁵ |
| rs16875079 | 8 | 107860271 | A | 9792 | 1.18 | (1.08-1.28) | 9.5 × 10⁻⁵ |
| rs12409323 | 1 | 38942258 | T | 4558 | 0.60 | (0.46-0.77) | 9.7 × 10⁻⁵ |

TABLE S10
Breakdown of minor allele frequencies for genome-wide significant SNPs.

| | Minor allele frequency | | | | | | | | |
|---|---|---|---|---|---|---|---|---|---|
| | | UK/Ire | UK | | German | German | | USA | USA |
| | UK/Ire | screened | population | German | screened | population | USA | screened | population |
| | cases | controls | controls | cases | controls | controls | cases | controls | controls |
| SNP | N = 2227 | N = 955 | N = 3881 | N = 555 | N = 37 | N = 787 | N = 1159 | N = 1086 | N = 1102 |
| rs2075650 | 0.30 | 0.13 | 0.15 | 0.30 | 0.18 | 0.16 | 0.32 | 0.15 | 0.14 |
| rs157580 | 0.30 | 0.42 | 0.38 | 0.27 | 0.38 | 0.35 | 0.26 | 0.39 | 0.38 |
| rs6859 | 0.52 | 0.42 | 0.42 | 0.52 | 0.45 | 0.45 | 0.51 | 0.43 | 0.44 |
| rs8106922 | 0.30 | 0.39 | 0.39 | 0.32 | 0.36 | 0.42 | 0.31 | 0.39 | 0.42 |
| rs405509 | 0.42 | 0.51 | 0.52 | 0.43 | 0.51 | 0.54 | 0.43 | 0.50 | 0.53 |
| rs10402271 | 0.38 | 0.30 | 0.32 | 0.40 | 0.30 | 0.33 | 0.41 | 0.31 | 0.32 |
| rs439401 * | 0.29 | 0.39 | 0.35 | 0.30 | 0.36 | 0.36 | 0.26 | 0.37 | 0.36 |
| rs2927438 | 0.24 | 0.21 | 0.22 | 0.26 | 0.26 | 0.22 | 0.26 | 0.20 | 0.21 |
| rs377702 | 0.42 | 0.35 | 0.38 | 0.43 | 0.42 | 0.41 | 0.44 | 0.38 | 0.41 |
| rs11136000 | 0.37 | 0.40 | 0.40 | 0.35 | 0.41 | 0.41 | 0.36 | 0.39 | 0.40 |
| rs1048699 | 0.12 | 0.09 | 0.09 | 0.11 | 0.14 | 0.09 | 0.11 | 0.08 | 0.09 |
| rs1114832 | 0.12 | 0.09 | 0.10 | 0.12 | 0.14 | 0.09 | 0.12 | 0.09 | 0.09 |
| rs1871047 | 0.37 | 0.41 | 0.40 | 0.35 | 0.39 | 0.37 | 0.34 | 0.40 | 0.39 |
| rs3851179 | 0.34 | 0.38 | 0.38 | 0.34 | 0.38 | 0.36 | 0.33 | 0.36 | 0.36 |
| rs5167 | 0.39 | 0.35 | 0.35 | 0.37 | 0.36 | 0.35 | 0.39 | 0.36 | 0.34 |

* This SNP was genotyped in a subsample of 3333 cases and 6460 controls.

TABLE S11
SNPs selected for follow-up genotyping. P-values in the GWAS, the extension sample, a previous
AD GWAS (TGEN), and the combined sample (Meta) are also shown. All p-values are two-tailed.

| | | | | | GWAS | Extension | TGEN | Meta | |
|---|---|---|---|---|---|---|---|---|---|
| | | | LD with GWS SNP | | P-value | P-value | P-value | P-value | Meta |
| SNP | Gene | Reason For Follow Up | D′ | r² | (N ≦ 11789) | (N ≦ 4233) | (N ≦ 1411) | (N ≦ 17433) | OR |
| rs7982 | CLU | Synonymous | 1.000 | 0.312 | 1 × 10⁻⁹ * | 0.032 | N/A | 8 × 10⁻¹⁰ † | 0.86 |
| rs3087554 | CLU | 3′UTR | 1.000 | 0.023 | N/A | 0.146 | N/A | 0.146 | 1.09 |
| rs9331888 | CLU | 5′UTR (transcript 2) | 1.000 | 0.468 | N/A | 0.304 | N/A | 0.304 | 1.05 |
| rs7012010 | CLU | GWAS P < 1 × 10⁻³ | 0.095 | 0.126 | 8 × 10⁻⁴ | 0.309 | 0.033 * | 1 × 10⁻⁴ † | 1.10 |
| rs561655 | PICALM | Within a Putative TFBS | 1.000 | 0.957 | 9 × 10⁻⁶ * | 0.016 | N/A | 1 × 10⁻⁷ † | 0.87 |
| rs592297 | PICALM | Synonymous | 0.199 | 0.708 | 6 × 10⁻⁵ * | 0.019 | 0.136 * | 2 × 10⁻⁷ † | 0.86 |
| rs636848 | PICALM | Within a Putative TFBS | 0.682 | 0.954 | 3 × 10⁻¹ * | 0.017 | N/A | 2 × 10⁻² † | 1.07 |
| rs532470 | PICALM | Putative eSNP | 0.100 | 0.590 | 7 × 10⁻² * | 0.498 | N/A | 3 × 10⁻² † | 1.06 |
| rs7941541 | PICALM | GWAS P < 1 × 10⁻⁴ | 0.960 | 0.875 | 2 × 10⁻⁷ | 0.189 | 0.005 * | 3 × 10⁻⁹ † | 0.86 |
| rs541458 | PICALM | GWAS P < 1 × 10⁻⁴ | 0.720 | 0.577 | 2 × 10⁻⁶ | 0.027 | 0.038 | 5 × 10⁻⁹ | 0.86 |
| rs543293 | PICALM | GWAS P < 1 × 10⁻⁴ | 0.923 | 0.910 | 7 × 10⁻⁷ | 0.109 | 0.023 | 1 × 10⁻⁸ | 0.87 |
| rs677909 | PICALM | GWAS P < 1 × 10⁻⁴ | 0.283 | 0.558 | 2 × 10⁻⁵ | 0.050 | 0.012 | 3 × 10⁻⁸ | 0.87 |

* P-value is based on imputed genotypes,
† Meta P-value is based on partially imputed genotypes.
GWS = genome-wide significant;
OR = odds ratio for the minor allele.

TABLE S12
Test of association between genome-wide significant SNPs and age at onset of Alzheimer's disease.

| | | Position | Number of | | |
|---|---|---|---|---|---|
| SNP | Chromosome | (basepairs) | individuals | β | P-value |
| rs2075650 | 19 | 50,087,459 | 2856 | −1.61 | 5.8 × 10⁻¹² |
| rs157580 | 19 | 50,087,106 | 2856 | 1.41 | 2.5 × 10⁻⁹ |
| rs6859 | 19 | 50,073,874 | 2854 | −0.80 | 1.8 × 10⁻⁴ |
| rs8106922 | 19 | 50,093,506 | 2851 | 0.63 | 0.006 |
| rs405509 | 19 | 50,100,676 | 2856 | 0.54 | 0.011 |
| rs10402271 | 19 | 50,021,054 | 2856 | −0.76 | 3.6 × 10⁻⁴ |
| rs439401 | 19 | 50,106,291 | 2428 | 1.39 | 2.5 × 10⁻⁷ |
| rs2927438 | 19 | 49,933,947 | 2852 | −0.61 | 0.012 |
| rs377702 | 19 | 50,054,507 | 2856 | −0.58 | 0.007 |
| rs11136000 | 8 | 27,520,436 | 2836 | −0.06 | 0.770 |
| rs1048699 | 19 | 50,342,226 | 2850 | −0.58 | 0.086 |
| rs1114832 | 19 | 50,328,041 | 2855 | −0.48 | 0.146 |
| rs1871047 | 19 | 50,043,586 | 2853 | 0.33 | 0.132 |
| rs3851179 | 11 | 85,546,288 | 2856 | 0.17 | 0.436 |
| rs5167 | 19 | 50,140,305 | 2856 | −0.13 | 0.554 |

TABLE S13
P-values for SNP × SNP interaction terms

| | rs744373 | rs11136000 | rs670139 | rs3851179 | rs3764650 | rs429358 |
|---|---|---|---|---|---|---|
| SNP | (BIN1) | (CLU) | (MS4A) | (PICALM) | (ABCAT) | (APOE) |
| rs3818361 (CR1) | 0.6607 | 0.4892 | 0.9616 | 0.1942 | 0.9913 | 0.9367 |
| rs744373 (BIN1) | | 0.9979 | 0.3080 | 0.9331 | 0.4509 | 0.1270 |
| rs11136000 (CLU) | | | 0.2964 | 0.0613 | 0.5545 | 0.6737 |
| rs670139 (MS4A) | | | | 0.0502 | 0.3671 | 0.6232 |
| rs3851179 (PICALM) | | | | | 0.4491 | 0.7350 |
| rs3764650 (ABCA7) | | | | | | 0.6242 |

NB: Data calculated from stage1 sample.

TABLE S14
Stage 1 P-values and details of SNP selection for Stage 2 genotyping and Stage 3 meta-anlaysis

| | | | Closest | Stage 1 | Stage 2 | |
|---|---|---|---|---|---|---|
| SNP | CHR | POSITION | Gene | P | P | Notes |
| rs3764650 | 19 | 997,520 | ABCA7 | 1.5E−06 | 1.3E−04 | Selected for Stage 3 meta-analysis |
| rs670139 | 11 | 59,728,371 | MS4A4E | 5.6E−06 | 9.2E−04 | Selected for Stage 3 meta-analysis |
| rs744373 | 2 | 127,611,085 | BIN1 | 2.0E−08 | 6.5E−03 | Selected for Stage 3 meta-analysis |
| rs3818361 | 1 | 205,851,591 | CR1 | 5.5E−12 | 8.9E−03 | Selected for Stage 3 meta-analysis |
| rs610932 | 11 | 59,695,883 | MS4A6A | 1.5E−06 | 2.8E−02 | Selected for Stage 3 meta-analysis |
| rs3135344 | 6 | 32,503,014 | HLA-DRA | 5.2E−06 | 2.3E−01 | Stage 2 P > 0.05, SNP not selected for Stage 3 |
| rs1539053 | 1 | 57,872,295 | DAB1 | 6.7E−06 | 2.6E−01 | Stage 2 P > 0.05, SNP not selected for Stage 3 |
| rs4782279 | 16 | 19,666,508 | IQCK | 1.3E−06 | 4.0E−01 | Stage 2 P > 0.05, SNP not selected for Stage 3 |
| rs9314866 | 9 | 79,907,769 | GNAQ | 8.2E−06 | 4.2E−01 | Stage 2 P > 0.05, SNP not selected for Stage 3 |
| rs7191155 | 16 | 19,707,714 | IQCK | 2.2E−06 | 4.2E−01 | Stage 2 P > 0.05, SNP not selected for Stage 3 |
| rs1858973 | 16 | 19,651,150 | IQCK | 4.9E−07 | 5.2E−01 | Stage 2 P > 0.05, SNP not selected for Stage 3 |
| rs7573507 | 2 | 68,882,220 | ARHGAP25 | 8.5E−06 | 6.8E−01 | Stage 2 P > 0.05, SNP not selected for Stage 3 |
| rs739565 | 16 | 19,624,006 | C16orf88 | 3.3E−06 | 7.2E−01 | Stage 2 P > 0.05, SNP not selected for Stage 3 |
| rs4958112 | 5 | 132,637,416 | FSTL4 | 1.6E−06 | 7.2E−01 | Stage 2 P > 0.05, SNP not selected for Stage 3 |
| rs4571225 | 3 | 191,807,148 | IL1RAP | 9.9E−06 | 8.1E−01 | Stage 2 P > 0.05, SNP not selected for Stage 3 |
| rs11767557 | 7 | 142,819,261 | EPHA1 | 7.2E−06 | 8.4E−01 | Stage 2 P > 0.05, SNP not selected for Stage 3 |
| rs3809278 | 12 | 110,209,568 | CUX2 | 5.3E−06 | 9.1E−01 | Stage 2 P > 0.05, SNP not selected for Stage 3 |
| rs10761558 | 10 | 62,193,476 | CDK1 | 1.2E−06 | 9.2E−01 | Stage 2 P > 0.05, SNP not selected for Stage 3 |
| rs8055533 | 16 | 10,949,740 | CLEC16A | 2.8E−06 | 9.5E−01 | Stage 2 P > 0.05, SNP not selected for Stage 3 |
| rs10501927 | 11 | 99,262,939 | CNTN5 | 1.6E−07 | 9.9E−01 | Stage 2 P > 0.05, SNP not selected for Stage 3 |
| rs11894266 | 2 | 170,344,888 | SSB | 5.9E−06 | N/A | SNP assay failed optimisation; rs13010581, a proxy SNP |
| | | | | | | (r² = 1) was genotyped in GERAD2 (P = 0.682) |
| rs1408077 | 1 | 205,870,764 | CR1 | 7.0E−11 | N/A | SNP was not selected for Stage 2 as a proxy SNP rs3818361 |
| | | | | | | (r² = 0.956) was genotyped through GERAD2 (P = 0.009) |
| rs6701713 | 1 | 205,852,912 | CR1 | 5.2E−11 | N/A | SNP was not selected for Stage 2 as a proxy SNP rs3818361 |
| | | | | | | (r² = 1) was genotyped through GERAD2 (P = 0.009) |
| rs667897 | 11 | 59,693,555 | MS4A6A | 3.0E−06 | N/A | SNP was not selected for Stage 2 as a proxy SNP rs610932 |
| | | | | | | (r² = 0.88) was genotyped through GERAD2 (P = 0.028) |
| rs2847666 | 11 | 59,616,152 | MS4A2 | 4.4E−06 | N/A | SNP was not selected for Stage 2 as it is in LD with rs610932 |
| | | | | | | (r² = 0.75) which was genotyped through GERAD2 (P = 0.028) |
| rs676309 | 11 | 59,758,149 | MS4A4E | 6.3E−07 | N/A | SNP was not selected for Stage 2 as a proxy SNP rs670139 |
| | | | | | | (r² = 1) was genotyped through GERAD2 (P = 0.0009) |
| rs405509 | 19 | 50,100,676 | APOE | 1.0E−16 | N/A | SNP was not selected for Stage 2 as it is located at the APOE locus |
| rs3760627 | 19 | 50,149,020 | CLPTM1 | 1.1E−08 | N/A | SNP was not selected for Stage 2 as it is located at the APOE locus |
| rs5167 | 19 | 50,140,305 | APOC4 | 5.7E−09 | N/A | SNP was not selected for Stage 2 as it is located at the APOE locus |
| rs1871045 | 19 | 50,018,608 | BCAM | 5.6E−06 | N/A | SNP was not selected for Stage 2 as it is located at the APOE locus |
| rs10402271 | 19 | 50,021,054 | BCAM | 1.0E−16 | N/A | SNP was not selected for Stage 2 as it is located at the APOE locus |
| rs8103315 | 19 | 49,946,008 | BCL3 | 6.4E−12 | N/A | SNP was not selected for Stage 2 as it is located at the APOE locus |
| rs2927438 | 19 | 49,933,947 | BCL3 | 1.5E−14 | N/A | SNP was not selected for Stage 2 as it is located at the APOE locus |
| rs4803750 | 19 | 49,939,467 | BCL3 | 2.7E−07 | N/A | SNP was not selected for Stage 2 as it is located at the APOE locus |
| rs2927488 | 19 | 49,923,318 | CEACAM16 | 6.4E−07 | N/A | SNP was not selected for Stage 2 as it is located at the APOE locus |
| rs2965101 | 19 | 49,929,652 | BCL3 | 4.8E−14 | N/A | SNP was not selected for Stage 2 as it is located at the APOE locus |
| rs7257916 | 19 | 50,174,724 | CLPTM1 | 6.8E−06 | N/A | SNP was not selected for Stage 2 as it is located at the APOE locus |
| rs8111069 | 19 | 50,175,278 | CLPTM1 | 2.7E−07 | N/A | SNP was not selected for Stage 2 as it is located at the APOE locus |
| rs2627641 | 19 | 50,400,598 | EXOC3L2 | 3.9E−06 | N/A | SNP was not selected for Stage 2 as it is located at the APOE locus |
| rs597668 | 19 | 50,400,728 | EXOC3L2 | 5.6E−07 | N/A | SNP was not selected for Stage 2 as it is located at the APOE locus |
| rs439401 | 19 | 50,106,291 | APOE | 1.0E−16 | N/A | SNP was not selected for Stage 2 as it is located at the APOE locus |
| rs1114832 | 19 | 50,328,041 | LRRC68 | 2.2E−11 | N/A | SNP was not selected for Stage 2 as it is located at the APOE locus |
| rs1048699 | 19 | 50,342,226 | LRRC68 | 4.2E−10 | N/A | SNP was not selected for Stage 2 as it is located at the APOE locus |
| rs7255066 | 19 | 49,837,943 | PVR | 9.8E−07 | N/A | SNP was not selected for Stage 2 as it is located at the APOE locus |
| rs1871047 | 19 | 50,043,586 | PVRL2 | 9.6E−13 | N/A | SNP was not selected for Stage 2 as it is located at the APOE locus |
| rs377702 | 19 | 50,054,507 | PVRL2 | 3.2E−11 | N/A | SNP was not selected for Stage 2 as it is located at the APOE locus |
| rs12610605 | 19 | 50,062,678 | PVRL2 | 1.8E−09 | N/A | SNP was not selected for Stage 2 as it is located at the APOE locus |
| rs11667640 | 19 | 50,071,631 | PVRL2 | 3.4E−06 | N/A | SNP was not selected for Stage 2 as it is located at the APOE locus |
| rs6859 | 19 | 50,073,874 | PVRL2 | 1.0E−16 | N/A | SNP was not selected for Stage 2 as it is located at the APOE locus |
| rs157580 | 19 | 50,087,106 | TOMM40 | 1.0E−16 | N/A | SNP was not selected for Stage 2 as it is located at the APOE locus |
| rs2075650 | 19 | 50,087,459 | TOMM40 | 1.0E−16 | N/A | SNP was not selected for Stage 2 as it is located at the APOE locus |
| rs8106922 | 19 | 50,093,506 | TOMM40 | 1.0E−16 | N/A | SNP was not selected for Stage 2 as it is located at the APOE locus |
| rs11136000 | 8 | 27,520,436 | CLU | 4.4E−16 | N/A | SNP was not selected for Stage 2 as it is located at the CLU locus |
| rs1237999 | 11 | 85,492,678 | PICALM | 3.6E−06 | N/A | SNP was not selected for Stage 2 as it is located at the PICALM locus |
| rs543293 | 11 | 85,497,725 | PICALM | 9.4E−08 | N/A | SNP was not selected for Stage 2 as it is located at the PICALM locus |
| rs659023 | 11 | 85,502,507 | PICALM | 4.5E−07 | N/A | SNP was not selected for Stage 2 as it is located at the PICALM locus |
| rs7941541 | 11 | 85,536,186 | PICALM | 9.1E−08 | N/A | SNP was not selected for Stage 2 as it is located at the PICALM locus |
| rs3851179 | 11 | 85,546,288 | PICALM | 7.5E−09 | N/A | SNP was not selected for Stage 2 as it is located at the PICALM locus |
| rs677909 | 11 | 85,435,237 | PICALM | 8.7E−08 | N/A | SNP was not selected for Stage 2 as it is located at the PICALM locus |
| rs536841 | 11 | 85,465,472 | PICALM | 1.7E−08 | N/A | SNP was not selected for Stage 2 as it is located at the PICALM locus |
| rs541458 | 11 | 85,465,999 | PICALM | 2.9E−08 | N/A | SNP was not selected for Stage 2 as it is located at the PICALM locus |

TABLE S15
Results for the 5 SNPs analysed in Stage 3 meta-analysis

| | | | | Closest | | | | Lower | Upper | | Cochran's | |
|---|---|---|---|---|---|---|---|---|---|---|---|---|
| Dataset | SNP | CHR | BP | gene | A1 | A2 | OR | 95% CI | 95% CI | P | Q test P | I² |
| GERAD1 | rs3764650 | 19 | 997,520 | ABCA7 | G | T | 1.25 | 1.13 | 1.38 | 1.6E−05 | | |
| ADNI | rs3764650 | 19 | 997,520 | ABCA7 | G | T | 1.01 | 0.58 | 1.75 | 9.7E−01 | | |
| TGEN | rs3764650 | 19 | 997,520 | ABCA7 | N/A | N/A | N/A | N/A | N/A | N/A | | |
| EADI1 | rs3764650 | 19 | 997,520 | ABCA7 | G | T | 1.21 | 1.08 | 1.37 | 4.0E−03 | | |
| GERAD2 | rs3764650 | 19 | 997,520 | ABCA7 | G | T | 1.27 | 1.12 | 1.43 | 1.3E−04 | | |
| CHARGE | rs3764650 | 19 | 997,520 | ABCA7 | G | T | 1.02 | 0.83 | 1.26 | 8.6E−01 | | |
| Meta-analysis | rs3764650 | 19 | 997,520 | ABCA7 | G | T | 1.22 | 1.14 | 1.30 | 7.3E−10 | 0.43 | 0% |
| (Fixed effects) | | | | | | | | | | | | |
| GERAD1 | rs670139 | 11 | 59,728,371 | MS4A4E | T | G | 1.13 | 1.06 | 1.20 | 1.0E−04 | | |
| ADNI | rs670139 | 11 | 59,728,371 | MS4A4E | T | G | 1.08 | 0.78 | 1.49 | 6.6E−01 | | |
| TGEN | rs670139 | 11 | 59,728,371 | MS4A4E | T | G | 1.27 | 1.08 | 1.49 | 3.0E−03 | | |
| EADI1 | rs670139 | 11 | 59,728,371 | MS4A4E | T | G | 1.06 | 0.98 | 1.14 | 1.2E−01 | | |
| GERAD2 | rs670139 | 11 | 59,728,371 | MS4A4E | T | G | 1.14 | 1.06 | 1.23 | 9.2E−04 | | |
| CHARGE_nonimputed | rs670139 | 11 | 59,728,371 | MS4A4E | T | G | 1.12 | 1.01 | 1.24 | 3.6E−02 | | |
| Meta-analysis | rs670139 | 11 | 59,728,371 | MS4A4E | T | G | 1.12 | 1.08 | 1.16 | 1.2E−09 | 0.43 | 0% |
| (Fixed effects) | | | | | | | | | | | | |
| GERAD1 | rs610932 | 11 | 59,695,883 | MS4A6A | T | G | 0.87 | 0.82 | 0.92 | 1.4E−06 | | |
| ADNI | rs610932 | 11 | 59,695,883 | MS4A6A | T | G | 0.88 | 0.64 | 1.22 | 4.5E−01 | | |
| TGEN | rs610932 | 11 | 59,695,883 | MS4A6A | N/A | N/A | N/A | N/A | N/A | N/A | | |
| EADI1 | rs610932 | 11 | 59,695,883 | MS4A6A | T | G | 0.93 | 0.86 | 1.00 | 4.6E−02 | | |
| GERAD2 | rs610932 | 11 | 59,695,883 | MS4A6A | T | G | 0.92 | 0.85 | 0.99 | 2.8E−02 | | |
| CHARGE_nonimputed | rs610932 | 11 | 59,695,883 | MS4A6A | T | G | 0.97 | 0.89 | 1.06 | 4.8E−01 | | |
| Meta-analysis | rs610932 | 11 | 59,695,883 | MS4A6A | T | G | 0.91 | 0.88 | 0.94 | 1.1E−07 | 0.30 | 18% |
| (Fixed effects) | | | | | | | | | | | | |
| GERAD1 | rs744373 | 2 | 127,611,085 | BIN1 | G | A | 1.17 | 1.09 | 1.25 | 3.2E−06 | | |
| ADNI | rs744373 | 2 | 127,611,085 | BIN1 | G | A | 1.13 | 0.83 | 1.56 | 4.4E−01 | | |
| TGEN | rs744373 | 2 | 127,611,085 | BIN1 | N/A | N/A | N/A | N/A | N/A | N/A | | |
| EADI1 | rs744373 | 2 | 127,611,085 | BIN1 | G | A | 1.15 | 1.06 | 1.25 | 4.0E−04 | | |
| GERAD2 | rs744373 | 2 | 127,611,085 | BIN1 | G | A | 1.12 | 1.03 | 1.21 | 6.5E−03 | | |
| CHARGE_nonimputed | rs744373 | 2 | 127,611,085 | BIN1 | G | A | 1.12 | 1.04 | 1.21 | 4.6E−03 | | |
| Meta-analysis | rs744373 | 2 | 127,611,085 | BIN1 | G | A | 1.14 | 1.10 | 1.19 | 2.1E−12 | 0.92 | 0% |
| (Fixed effects) | | | | | | | | | | | | |
| GERAD1 | rs3818361 | 1 | 205,851,591 | CR1 | A | G | 1.17 | 1.09 | 1.25 | 9.2E−06 | | |
| ADNI | rs3818361 | 1 | 205,851,591 | CR1 | A | G | 1.58 | 1.06 | 2.35 | 2.4E−02 | | |
| TGEN | rs3818361 | 1 | 205,851,591 | CR1 | N/A | N/A | N/A | N/A | N/A | N/A | | |
| EADI1 | rs3818361 | 1 | 205,851,591 | CR1 | A | G | 1.28 | 1.17 | 1.40 | 8.5E−08 | | |
| GERAD2 | rs3818361 | 1 | 205,851,591 | CR1 | A | G | 1.13 | 1.03 | 1.25 | 8.9E−03 | | |
| CHARGE | rs3818361 | 1 | 205,851,591 | CR1 | A | G | 1.04 | 0.94 | 1.16 | 4.7E−01 | | |
| Meta-analysis | rs3818361 | 1 | 205,851,591 | CR1 | A | G | 1.17 | 1.12 | 1.22 | 1.9E−12 | | |
| (Fixed effects) | | | | | | | | | | | | |
| Meta-analysis | rs3818361 | 1 | 205,851,591 | CR1 | A | G | 1.17 | 1.08 | 1.26 | 1.2E−04 | 0.02 | 64% |
| (Random effects) | | | | | | | | | | | | |

TABLE S16
Flanking sequence (bases) of each SNP of interest.

| | Closest | |
|---|---|---|
| SNP | Gene | Flanking Sequence |
| rs11136000 | CLU | agccacaccagctatcaaaa[T/C]tctctaacgggcccttgcca (SEQ ID NO: 1) |
| rs7982 | CLU | gccatggacatccacttcca[T/C]agcccggccttccagcaccc (SEQ ID NO: 2) |
| rs3851179 | PICALM | gttatgtgtgaagtcattta[T/C]aatagatagtgttgataata (SEQ ID NO: 3) |
| rs561655 | PICALM | gttaacctgggagtgaacta[G/A]acattaaaggggcagcatac (SEQ ID NO: 4) |
| rs592297 | PICALM | aaatcaagaagtgcatccat[C/T]tgattctgaataattggtac (SEQ ID NO: 5) |
| rs1408077 | CR1 | tctcagtagtggggttttgt[C/A]acctttactgttattattag (SEQ ID NO: 6) |
| rs6701713 | CR1 | tgcctgcagcccaacagatg[G/A]cagtgtgcttaacagctctg (SEQ ID NO: 7) |
| rs3818361 | CR1 | tataccccgttaaaggaaac[G/A]atatagaatacgaatggtct (SEQ ID NO: 8) |
| rs7561528 | BIN1 | tagtttcaagtaaacatgtc[G/A]cagtgaagtttgttgtagag (SEQ ID NO: 9) |
| rs744373 | BIN1 | ccctgtccgtccagactccg[A/G]gagtctccgacgggtactac (SEQ ID NO: 10) |
| rs3764650 | ABCA7 | caggctgcgaactttgcacc[T/G]ttacaccactccacgtgacc (SEQ ID NO: 11) |
| rs1562990 | MS4A4A | caccacacacaaggcctgaa[C/A]gatcaaaggctgaagagatg (SEQ ID NO: 12) |
| rs667897 | MS4A6A | cgctccaaacccgctgtgtc[A/G]taccataccggatgtttacc (SEQ ID NO: 13) |
| rs676309 | MS4A4E | aatggtgagataggtataga[A/G]tagactgtctcagttcaaaa (SEQ ID NO: 14) |
| rs583791 | MS4A6A | ggcaaagaggggaggaagat[G/A]ccaatagcttagattccca (SEQ ID NO: 15) |
| rs662196 | MS4A6A | gtttgggcatctgggggaaa[G/A]ccaggtttatgtaaatcaaa (SEQ ID NO: 16) |
| rs610932 | MS4A6A | agtctgaatttccagaaaac[A/C]atgatcattcaatggatcac (SEQ ID NO: 17) |
| rs670139 | MS4A4E | atctccaagtcaaagtttac[C/A]tcaagttgggccaatccctg (SEQ ID NO: 18) |

NB: The position of the SNP of interest within the flanking sequence is depicted by the square brackets. The nucleotide change at this SNP is displayed within the square brackets.

### REFERENCES

1. Abraham, R. A genome-wide association study for late-onset Alzheimer's disease using DNA pooling. BMC Med. Genomics 1, 44 (2008).
2. Beecham, G. Genome-wide association study implicates a chromosome 12 risk locus for late-onset Alzheimer disease. Am. J. Hum. Genet. 84, 35-43 (2009).
3. Bertram, L. Genome-wide association analysis reveals putative Alzheimer's disease susceptibility loci in addition to APOE. Am. J. Hum. Genet. 83, 623-632 (2008).
4. Carrasquillo, M. Genetic variation in PCDH11X is associated with susceptibility to late-onset Alzheimer's disease. Nat. Genet. 41, 192-198 (2009).
5. Coon, K. A high-density whole-genome association study reveals that APOE is the major susceptibility gene for sporadic late-onset Alzheimer's disease. J. Clin. Psychiatry 68, 613-618 (2007).
6. Grupe, A. Evidence for novel susceptibility genes for late-onset Alzheimer's disease from a genome-wide association study of putative functional variants. Hum. Mol. Genet. 16, 865-873 (2007).
7. Li, H. Candidate single-nucleotide polymorphisms from a genomewide association study of Alzheimer disease. Arch. Neurol. 65, 45-53 (2008).
8. Reiman, E. GAB2 alleles modify Alzheimer's risk in APOE epsilon4 carriers. Neuron 54, 713-720 (2007).
9. Harold, D. et al. Genome-wide association study identifies variants at CLU and PICALM associated with Alzheimer's disease. Nat Genet 41, 1088-1093 (2009).
10. Hollingworth, P. et al. Evidence that ABCA7 and MS4A are novel susceptibility loci for Alzheimer's disease and further support for BIN1 and CR1. Nature Genetics Submitted,
11. Wichmann, H., Gieger, C. & Illig, T. KORA-gen-resource for population genetics, controls and a broad spectrum of disease phenotypes. Gesundheitswesen 67 Suppl 1, S26-30 (2005).
12. Birnbaum, S. et al. Key susceptibility locus for nonsyndromic cleft lip with or without cleft palate on chromosome 8q24. Nat. Genet 41, 473-477 (2009).
13. Hillmer, A. M. et al. Susceptibility variants for male-pattern baldness on chromosome 20p11. Nat. Genet 40, 1279-1281 (2008).
14. Brouwers, N. et al. Genetic variability in progranulin contributes to risk for clinically diagnosed Alzheimer disease. Neurology 71, 656-664 (2008).
15. Teo, Y. Y. et al. A genotype calling algorithm for the Illumina BeadArray platform. Bioinformatics 23, 2741-2746 (2007).
16. Clayton, D. G. et al. Population structure, differential bias and genomic control in a large-scale, case-control association study. Nat Genet 37, 1243-1246 (2005).
17. Moskvina, V., Craddock, N., Holmans, P., Owen, M. J. & O&rsquo; Donovan, M. C. Effects of Differential Genotyping Error Rate on the Type I Error Probability of Case-Control Studies. Hum Hered 61, 55-64 (2006).
18. Purcell, S. et al. PLINK: A Tool Set for Whole-Genome Association and Population-Based Linkage Analyses. The American Journal of Human Genetics 81, 559-575 (2007).
19. Price, A. L. et al. Long-Range LD Can Confound Genome Scans in Admixed Populations. The American Journal of Human Genetics 83, 132-135 (2008).
20. Price, A. L. et al. Principal components analysis corrects for stratification in genome-wide association studies. Nat. Genet 38, 904-909 (2006).
21. Devlin, B. & Roeder, K. Genomic control for association studies. Biometrics 55, 997-1004 (1999).
22. Reiman, E. M. et al. GAB2 alleles modify Alzheimer's risk in APOE epsilon4 carriers. Neuron 54, 713-720 (2007).
23. Potkin, S. G. et al. Hippocampal atrophy as a quantitative trait in a genome-wide association study identifying novel susceptibility genes for Alzheimer's disease. PLoS ONE 4, e6501 (2009).
24. Lambert, J. et al. Genome-wide association study identifies variants at CLU and CR1 associated with Alzheimer's disease. Nat. Genet 41, 1094-1099 (2009).
25. Seshadri, S. et al. Genome-wide analysis of genetic loci associated with Alzheimer disease. JAMA 303, 1832-1840 (2010).
26. Conde, L. et al. PupaSuite: finding functional single nucleotide polymorphisms for large-scale genotyping purposes. Nucleic Acids Res 34, W621-625 (2006).

1. A method for screening for or diagnosing the likelihood of developing Alzheimer's disease, comprising:
(a) providing a tissue sample which has been extracted from a human body of an individual to be tested wherein the tissue sample contains at least a locus containing the clusterin (CLU), also known as APOJ, gene on chromosome 8, the PICALM gene on chromosome 11; complement receptor 1 gene (CR1) on chromosome 1; the bridging integrator/gene (BIN1) on chromosome 2; the ATP-binding cassette, sub family A, member 7 (ABCA7) on chromosome 19 and the membrane-spanning 4A (MS4A) gene cluster on chromosome 11;
(b) examining said locus in order to identify whether SNP rs11136000 is present in the clusterin locus; rs3851179 is present in the PICALM locus; rs1408077, rs6701713 or rs3818361 is present in the CR1 locus; rs7561528 or rs744373 is present in the BIN1 locus; rs3764650 is present in the ABCA7 locus; and rs670139, rs610932, rs676309, rs667897, rs662196, rs583791 or rs1562990 is present in the MS4A gene cluster locus; and
(c) where SNP rs11136000 is present in the clusterin locus and rs3851179 is present in the PICALM locus; one of rs1408077, rs6701713 or rs3818361 is present in the CR1 locus; one of rs7561528 or rs744373 is present in the BIN1 locus; rs3764650 is present in the ABCA7 locus; and one of rs670139, rs610932, rs676309, rs667897, rs662196, rs583791 or rs1562990 is present in the MS4A gene cluster locus concluding that the individual from whom the sample has been extracted is likely to develop, or is suffering from, Alzheimer's disease.

2. The method according to claim 1 wherein:
in step (b) additionally, or alternatively, to examining for rs11136000 in said clusterin locus said locus is examined to identify whether rs7982 is present, and additionally, or alternatively, to examining for rs3851179 in said PICALM locus said locus is examined to identify whether rs561655 or rs592297 is present; and
in step (c) where rs7982, rs561655 or rs592297 is present concluding that the individual from whom the sample has been extracted is likely to develop, or is suffering from, Alzheimer's disease.

3. The method according to claim 1 wherein at least one labelled oligonucleotide, complementary to the genetic locus/loci or cluster to be examined, is used to detect said SNP(s), wherein said oligonucleotide upon binding to, and so detecting, said SNP, emits a detectable signal representative of the presence of said SNP.

4. The method according to claim 1 wherein said tissue sample is PCR amplified prior to performing step (b).

5. The method according to claim 1 wherein said tissue is enzymatically fragmented prior to performing step (b).

6. The method according to claim 3 wherein said complementary oligonucleotide is attached or bound to a solid phase or substrate and said tissue sample is exposed to said solid phase prior to performing step (b).

7. A kit for screening for or diagnosing the likelihood of developing Alzheimer's, disease comprising:
a plurality of oligonucleotides at least one of which is complementary to least one of a loci or gene cluster comprising the clusterin (CLU, also known as APOJ) gene on chromosome 8, the PICALM gene on chromosome 11, the complement receptor/gene (CR1) on chromosome 1, the bridging integrator 1 gene (BIN1) on chromosome 2, the ATP-binding cassette, sub family A, member 7 (ABCA7) on chromosome 19 or the membrane-spanning 4A (MS4A) gene cluster on chromosome 11;
wherein said oligonucleotide(s) is/are able to detect one or more of the following SNPs rs11136000 in CLU, rs3851179 in PICALM, rs1408077, rs6701713 or rs3818361 in CR1; rs7561528 or rs744373 in BIN); rs3764650 in ABCA7, and rs670139, rs610932, rs676309, rs667897, rs662196, rs583791 or rs1562990 in the MS4A gene cluster;
further wherein each of said plurality of oligonucleotides is provided with a label that emits a detectable signal upon binding to said SNP.

8. A kit for screening for or diagnosing the likelihood of developing Alzheimer's disease, comprising:
a plurality of oligonucleotides at least one of which is complementary to least one of a loci or gene cluster comprising the clusterin (CLU, also known as APOJ) gene on chromosome 8, the PICALM gene on chromosome 11, the complement receptor/gene (CR1) on chromosome 1, the bridging integrator 1 gene (BIN1) on chromosome 2, the ATP-binding cassette, sub family A, member 7 (ABCA7) on chromosome 19 or the membrane-spanning 4A (MS4A) gene cluster on chromosome 11;
wherein said oligonucleotide(s) is/are able to detect one or more of the following SNPs rs11136000 in CLU, rs3851179 in PICALM rs1408077, rs6701713 or rs3818361 in CR1; rs7561528 or rs744373 in BIN1; rs3764650 in ABCA7, and rs670139, rs610932, rs676309, rs667897, rs662196, rs583791 or rs1562990 in the MS4A gene cluster;
further wherein said kit includes labelling means which is used in combination with said oligonucleotide(s) whereby binding of the oligonucleotide to said SNP enables the labelling means to detect the aforementioned binding and so produce a signal representative of the presence of said SNP.

9. A kit according to claim 8 wherein said oligonucleotide(s) is/are immobilised on a solid support.

10. A nucleic acid molecule comprising a plurality of genetic loci or cluster comprising the clusterin (CLU, also known as APOJ) gene on chromosome 8, the PICALM gene on chromosome 11, the complement receptor/gene (CR1) on chromosome 1, the bridging integrator/gene (BIN1) on chromosome 2, the ATP-binding cassette, sub family A, member 7 (ABCA7) on chromosome 19 or the membrane-spanning 4A (MS4A) gene cluster on chromosome 11; and further including one or more of the following variants, including any combination thereof:
SNP rs11136000 or rs7982 in the clusterin locus and rs3851179 or rs561655 or rs592297 in the PICALM locus; one of rs1408077, rs6701713 or rs3818361 in the CR1 locus; one of rs7561528 or rs744373 in the BIN1 locus; rs3764650 in the ABCA7 locus; and one of rs670139, rs610932, rs676309, rs667897, rs662196, rs583791 or rs1562990 in the MS4A gene cluster locus

11. A cell or cell-line comprising a plurality of genetic loci or cluster comprising the clusterin (CLU, also known as APOJ) gene on chromosome 8, the PICALM gene on chromosome 11, the complement receptor/gene (CR1) on chromosome 1, the bridging integrator/gene (BIN1) on chromosome 2, the ATP-binding cassette, sub family A, member 7 (ABCA7) on chromosome 19 or the membrane-spanning 4A (MS4A) gene cluster on chromosome 11; and which further includes one or more of the following SNP rs11136000 or rs7982 in the clusterin locus and rs3851179 or rs561655 or rs592297 in the PICALM locus; one of rs1408077, rs6701713 or rs3818361 in the CR1 locus; one of rs7561528 or rs744373 in the BIN1 locus; rs3764650 in the ABCA7 locus; and one of rs670139, rs610932, rs676309, rs667897, rs662196, rs583791 or rs1562990 in the MS4A gene cluster locus

12. A cell or cell line according to claim 11 further comprising SNP rs7982; SNP rs561655; SNP rs592297.

13. A cell or cell line according to claim 11, comprising a functional endocytic, apoptotic, complement or innate immune response pathway.

14. A cell or cell line according to claim 12, comprising a functional endocytic, apoptic, complement or innate immune response pathway.

15. A method for testing whether a therapeutic, label or identification means can be used to treat, label or identify, respectively, Alzheimer's disease, comprising:
exposing a cell or cell line according to claim 14 to a test substance and observing:
a) the effect of said test substance on an endocytic, apoptic, complement or innate immune response pathway of said cell or cell line; or
b) observing the ability of said test substance to label or identify said cell or cell line by labelling or identifying said SNPs.

16. A method for testing whether a therapeutic, label or identification means can be used to treat, label or identify, respectively, Alzheimer's disease, comprising:
exposing a cell or cell line according to claim 15 to a test substance and observing:
a) the effect of said test substance on an endocytic, apoptic, complement or innate immune response pathway of said cell or cell line; or
b) observing the ability of said test substance to label or identify said cell or cell line by labelling or identifying said SNPs.
